(19) United States
(12) Patent Application Publication
(10) Pub. No.: US 2007/0060769 A1
(43) Pub. Date: Mar. 15, 2007

(54) Integrated process to produce derivatives of butadiene addition products

(75) Inventors: Benjamin Patrick Gracey, East Riding of Yorkshire (GB); Christopher Halletr, Hertfordshire (GB)

Correspondence Address: WELSH & KATZ, LTD, 120 S RIVERSIDE PLAZA, 22ND FLOOR, CHICAGO, IL, 60606, US

(73) Assignee: The University Of Southern Mississippi Research Foundation, Hattiesburg, MS (US)

(21) Appl. No.: 10/557,718

(22) PCT Filed: Jun. 15, 2004
(86) PCT No.: PCT/GB04/02508

§ 371 Date: Nov. 17, 2005

(30) Foreign Application Priority Data
Jun. 16, 2003 (GB) 0313920.1
Jun. 16, 2003 (GB) 0313919.3
Jun. 16, 2003 (GB) 0313918.5
Jun. 16, 2003 (GB) 0313908.6

(51) Int. Cl. C07C 67/00

(52) U.S. Cl. 560/241

(57) ABSTRACT

An integrated chemical process to form derivatives of butadiene addition products comprises forming an addition product of butadiene and a selected carboxylic acid, alcohol, or glycol, to form a reaction mixture containing at least a crotyl addition product and a sec-butenyl addition product; separating the reaction product mixture into streams comprising a crotyl product stream, a sec-butenyl product stream, and at least one stream containing other reacted and unreacted products; controlling the proportion of the product streams, preferably by recycling a portion or all of a separated crotyl product stream and/or a sec-butenyl product stream and other product streams to the addition reactor; subjecting one or more separated product streams to one or more process selected from hydrolysis, hydrogenation, and isomerization to form product derivatives in preselected proportions; and recovering one or more resulting product derivatives.

### BACKGROUND OF THE INVENTION

This invention relates to integrated processes for producing derivatives of reaction products of butadiene with a carboxylic acid or an alcohol or diol in the presence of an acidic or a Lewis acid catalyst. The invention further relates to integrated processes which may be controlled to produce a variety of saturated and unsaturated C₄ esters or ethers and derivatives including alcohols, aldehydes, and ketones in varying proportions.

Unsaturated butyl esters and ethers are valuable intermediates for producing chemicals such as butyl acetate, n-butanol, sec-butanol, allylic alcohols, butyraldehyde, monomers, butyl glycol ethers, butyl ethers, butyl glymes and methyl ethyl ketone. This invention is an integrated process to produce a variety of butadiene derivatives in a chemical process apparatus.

Butyraldehyde may be produced by a number of routes, for example by hydroformylation of propene (propylene). Other recently-proposed routes, e.g., U.S. Pat. No. 5,705,707, disclose a method of making butyraldehyde and n-butanol by reacting butadiene with an alcohol in the presence of an acidic catalyst to form a mixture of isomeric unsaturated ethers 3-alkoxybutene-1 and 1-alkoxybutene-2, isomerising the former to the latter followed by isomerisation to the enol form and hydrolysis.

Methyl ethyl ketone (“MEK”) is an important solvent with similar properties to those of acetone but with a lower evaporation rate. It finds use in the production of transparent paper, printing inks, synthetic leather, degreasing of metal surfaces; extraction of fats, lacquers; oils, waxes, natural resins; dewaxing of mineral oils. Butyraldehyde is an important chemical intermediate that is used in the manufacture of chemicals such as n-butanol, 2-ethylhexanol and trimethylol propane.

Methyl ethyl ketone can be produced by a number of known routes. Erdöl Informations-Dienst A. M. Stahmer, vol. 37, no. 28 (1984) discloses a process for making methyl ethyl ketone by the dehydrogenation of sec-butyl alcohol.

U.S. Pat. No. 3,196,182 discloses co-production of acetic acid and MEK by catalytic oxidation of butane. U.S. Pat. No. 3,215,734 and JP 46-2010 disclose production of MEK by the direct oxidation of n-butenes. DE-OS 2300903 discloses decomposition of sec-butylbenzenehydroperoxide to provide phenol and MEK. DE 935503 discloses that autoxidation of sec-butyl alcohol gives MEK and hydrogen peroxide.

n-Butyl esters, such as n-butyl acetate, may be produced by a number of known routes. For instance, hydroformylation of propylene in the presence of acetic acid produces a mixture of n-butyl acetate and iso-butyl acetate. This method however requires a source of syngas (CO+H₂), which increases capital costs. An alternative method is to react ethylene with vinyl acetate in the presence of an acid catalyst followed by the hydrogenation of the resultant unsaturated ester. A further method is the reaction of ethylene with ethanol in the presence of a base catalyst to form butanol, and the reaction of the produced butanol with acetic acid to form butyl acetate. In addition, all these methods rely on use of either relatively expensive feedstock such as ethylene and n-butanol, or involve multiple reaction stages, or expensive catalysts and separation stages. Acid catalysed addition of butadiene to acetic acid using ion-exchange resin catalysts having bulky counterions to improve the reaction selectivity to two isomeric C₄ butenyl acetates is described in U.S. Pat. Nos. 4,450,288, 4,450,287, and 4,450,289. These patents primarily are directed to production of secondary butenyl acetate.

Also known is that the addition reaction of butadiene to carboxylic acids may be catalysed by homogeneous catalysts, such as sulphonic acids (cf. WO03/082796), and mineral acids such as sulphuric acid (described in U.S. Pat. No. 6,465,683). In all cases a significant loss of selectivity based on butadiene is observed due to the formation of by-products. This patent also describes how the control of water level with ion-exchange reaction catalyst systems can improve the reaction selectivity and describes how recycle of secondary butenyl ester to the reactor can be conducted to improve the butadiene selectivity to the crotyl ester. Despite this, some selectivity loses still occur based on both the carboxylic acid and butadiene components.

WO03/020681 discloses reacting acetic acid with a mixed C₄ stream comprising iso-butene and 1,3-butadiene in an addition reactor, withdrawing a product stream comprising iso-butene, sec-butenyl acetate, n-butenyl acetate and t-butyl acetate and recycling the t-butyl acetate to the addition reactor. This suppresses further reaction of the isobutene and increases the selectivity on carboxylic acid.

Unsaturated ethers, such as butenyl ethers may be prepared by a variety of different methods. Alkyl ethers, for example, n-butyl glycol ether, have been produced commercially by the reaction of an alkanol with an olefin oxide such as e.g. ethylene oxide. However, such a process leads to the formation of a significant amount of unwanted by-products, for example, diglycol ethers. The presence of by-products adds complexity to the separation of the desired alkyl mono ethers of glycols and can adversely affect the process economics. It is also known that butadiene can be reacted with an alcohol to form a mixture of isomeric unsaturated ethers. U.S. Pat. No. 2,922,822 discloses an earlier method of making butenyl ethers by reacting butadiene with an alcohol in the presence of an acidic ion-exchange resin catalyst. A similar process also is disclosed in DE-A-2550902.

Butadiene is a relatively inexpensive by-product of hydrocarbon refining processes and is a potential feedstock for making butyl esters and ethers. It is commercially available either as a purified chemical or as a constituent of a hydrocarbon cut. For example, as a constituent of a mixed C₄ stream derived from naphtha steam cracking operations such a crude C₄ stream contains species such as butane, 1-butene, 2-butene, isobutane, and isobutene in addition to butadiene. It is advantageous that a process using butadiene can use such mixed streams.

However, butadiene also is in thermal equilibrium with 4-vinyl cyclohexene, a Diels Alder dimer of butadiene. This dimer can be thermally cracked back to butadiene:

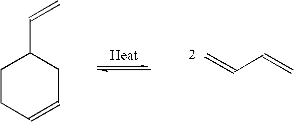

Thus, a process involving the use of a butadiene feedstock needs to take this reversible reaction into consideration and this is often achieved by recycle of this material to the carboxylic acid or alcohol addition reactor.

Similarly when a crude C₄ stream from a steam cracker is used instead of butadiene, recycle of t-butyl ester, formed from the equilibrium limited addition reaction of isobutene to the carboxylic acid, may be used to suppress the forward reaction of isobutene resulting in the formation of a stream rich in isobutene commonly referred to as raffinate 1.

For example, when an n-butyl ester such as butyl acetate is the desired reaction product from the reaction of crude C₄'s with a carboxylic acid, recycle of both the t-butyl and secondary-butenyl ester can be employed e.g.

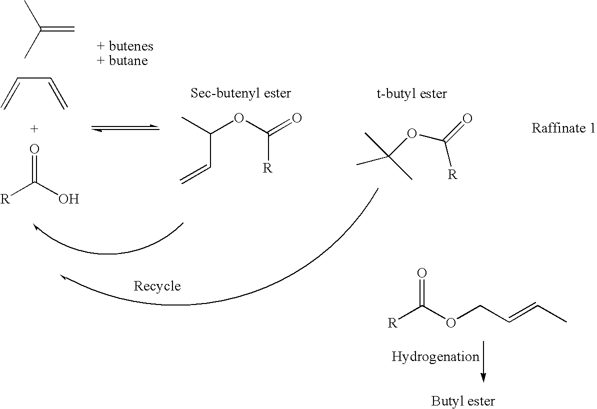

The other reaction by-products are commonly oligomers of butadiene which may have the carboxylic acid or alcohol moiety incorporated and the formation of these materials currently represents a lost of selectivity on both the butadiene feedstock and in some species of the carboxylic acid or alcohol feedstock.

In an attempt to reduce the formation of by-products, DE-A-4431528 describes a process, which involves the use of amines. In this document, a three/four step process is proposed comprising addition of an amine to butadiene, isomerisation of the addition product to an enamine, hydrolysis of the enamine to give butyraldehyde that may be optionally hydrogenated to the corresponding alcohol, if desired.

U.S. Pat. No. 6,403,839 describes a process for making n-butyraldehyde and methyl ethyl ketone comprising addition of a carboxylic acid to butadiene to form a mixture of crotyl ester and sec-butenyl ester in equilibrium:

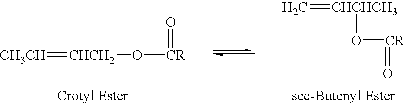

As noted above, the art contains many different processes to form the butadiene derivatives which may be produced in the integrated process of this invention. In a process operated according to this invention, a variety of starting materials may be used, such as pure butadiene and butadiene contained in a mixed C₄ refinery stream, which may be reacted with carboxylic acids or alcohols (including polyhydoxyl compounds such as glycols) and then further processed to form desired products in controlled proportions.

For example, addition of butadiene to carboxylic acids produces two isomeric C₄ derivatives—sec-butenyl ester that can be converted to MEK and crotyl ester that can be converted to butyraldehyde. Accordingly, MEK and butyraldehyde can be co-produced with the advantages inherent in economies of scale. Further, recycle of the sec-butenyl or the crotyl derivative or a mixture enriched in one isomer to the butadiene addition reaction stage allows facile control of the relative amounts of MEK and butyraldehyde produced.

Addition of butadiene to carboxylic acids or alcohols also provides an attractive alternative as a source of methyl ethyl ketone, butyraldehyde and other downstream products and a significant feedstock cost advantage to the new process and use of impure butadiene raffinate streams may further reduce feedstock costs.

There is a need for an efficient process which is capable of producing a selection of derivatives of primary and secondary butenyl alcohols, ethers and glycols produced by direct addition of butadiene with a reactive species. There is an especial need to produce a variety of such products in a single manufacturing unit comprising addition reactors, separation facilities and sections capable of forming isomerisation, hydrolysis, and hydrogenation functions, which forms an integrated process. Further, recycle of unreacted and by-products produces an efficient process without environmentally detrimental waste streams.

### SUMMARY OF THE INVENTION

An integrated chemical process to form derivatives of butadiene addition products comprises forming an addition product of butadiene and a selected carboxylic acid, alcohol, or glycol, to form a reaction mixture containing at least a crotyl addition product and a sec-butenyl addition product; separating the reaction product mixture into streams comprising a crotyl product stream, a sec-butenyl product stream, and at least one stream containing other reacted and unreacted products; controlling the amounts of crotyl addition product, sec-butenyl addition product and unreacted products in the reaction mixture by subjecting at least a portion of said separated streams to reaction conditions in which unreacted reactants and products undergo further reaction with one another; subjecting one or more separated product streams to one or more process selected from hydrolysis, hydrogenation, and isomerization to form product derivatives in preselected proportions; and recovering one or more resulting product derivatives. In preferred embodiments the amounts of crotyl addition product, sec-butenyl addition product and an unreacted products are determined by recycling selected proportions of the product streams. I.e. a portion or all of a separated crotyl product stream and/or a sec-butenyl product stream and other product streams may be recycled to the addition reactor.

### BRIEF DESCRIPTION OF THE DRAWINGS

FIG. 1 is a schematic representation of an integrated process to produce possible oxygenated products according to this invention.

FIG. 2 is a schematic representation of an integrated process to produce butanol and butyl carboxylate according to this invention.

FIG. 3 is a schematic representation of an integrated process to produce butyraldehyde, n-butanol, and 2-ethylhexanol according to this invention.

FIG. 4 is a schematic representation of an integrated process to co-produce butyraldehyde and methyl ethyl ketone according to this invention.

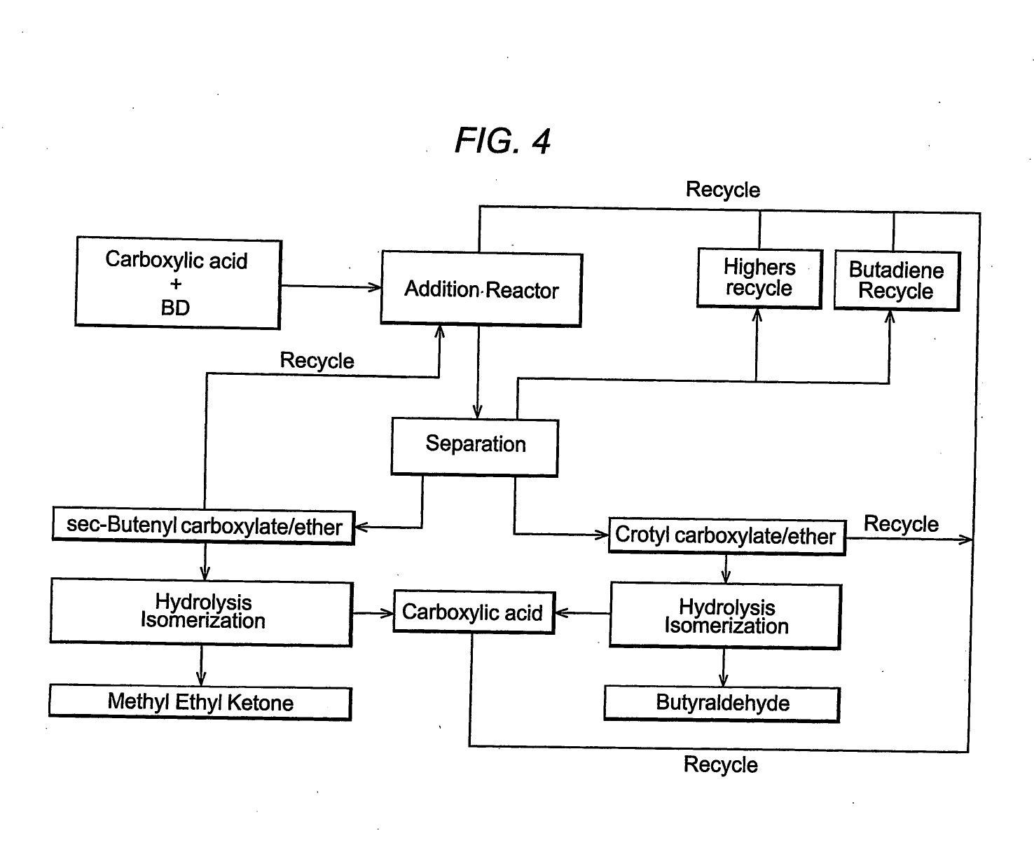

FIG. 5 is a gas chromatogram of a typical catalytic reaction addition product of butadiene and acetic acid according to this invention.

FIG. 6 is a gas chromatogram of a concentrated by-product mixture from Example 1.

### DESCRIPTION OF THE INVENTION

In the process of this invention, a hydrocarbon stream containing a conjugated diene such as butadiene is contacted with a reactive compound, Q, under addition conditions and the reaction products separated, recycled, and further converted to constitute an integrated process to produce butadiene derivatives.

The conjugated diene employed in the present invention is suitably a C₄ to C₁₀ aliphatic diene. Examples of suitable dienes are 1,3-butadiene, 1,3-pentadiene, 2-methyl-1,3-butadiene (isoprene). The most preferred diene is 1,3-butadiene (butadiene). The diene may be used in substantially pure form or in a hydrocarbon mixture. Butadiene is a relatively inexpensive by-product of hydrocarbon refining processes and is commercially available either as a purified chemical or as a constituent of a hydrocarbon cut. For example, butadiene is a constituent of a mixed C₄ stream containing compounds such as butane, 1-butene, 2-butene, isobutane, and isobutene. Advantageously, a process using butadiene uses such streams. Typically, up to about 60 wt. % of butadiene is present in such streams, although higher or lower concentrations may be useful in this process.

The process of the present invention provides an improved process for the production of a variety of chemicals, for example, the direct products, crotyl derivatives and secondary but-3-enyl derivatives which are, for example, carboxylates, ethers or glycol ethers. Such products can be converted to other useful products, for example, butyraldehyde, n-butanol, butyl esters, butyl ethers and butyl glycol ethers. The process can also be used for the removal of butadiene from refinery streams, particularly C₄ streams.

The reaction of butadiene with a carboxylic acid, an alcohol, including mono-, di-, and trihydric alcohols, provides an alternative entry to butyl derivatives currently provided by hydroformylation of propene known as the OXO process. Currently the major route to butyl derivatives is by the hydroformylation of propene to butyraldehyde, followed by hydrogenation to yield n-butanol. Butyraldehyde also is a valuable intermediate for materials such as 2-ethylhexanol and trimethylol propane.

In an aspect of this invention, alkylene ethers, especially alkylene glycol ethers, can be synthesised by an improved process employing certain homogeneous sulphonic acid catalysts.

In one aspect of this invention, a single chemical integrated process unit is capable of producing such a variety of useful commercial chemical products. Such an integrated unit typically would comprise a butadiene addition reactor, a primary product separation unit, a hydrolysis unit, a hydrogenation unit, and an isomerization unit together with primary and recycle piping and control units. Advantageously, some units may be combined in a single facility such as combining isomerization and hydrolysis. An advantage of such an integrated facility is flexibility in selecting the quantities of desired products produced from such facility. Using the same unit to produce such a variety of end products increases the overall usage of an efficiently scaled unit and permits production of a selection of lower volume products.

One aspect of this invention is an integrated chemical process comprising

(a) combining a hydrocarbon stream comprising butadiene with a compound Q selected from compounds defined as:
R¹(CO)ₙ—OH
wherein n is 1 or 0, and
R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups,
   under addition reaction conditions to form a reaction mixture containing at least a crotyl addition product and a sec-butenyl addition product;

(b) separating the reaction product mixture into streams comprising a crotyl product containing stream, a sec-butenyl containing product stream, and at least one stream containing other reacted and unreacted products;

(c) controlling the proportion of the product streams by recycling a portion or all of a separated crotyl product stream and/or a sec-butenyl product stream and other product streams to the addition reactor;

(d) subjecting one or more separated product streams to one or more process selected from hydrolysis, hydrogenation, and isomerization to form product derivatives in preselected proportions; and

(e) recovering one or more resulting product derivatives.

An aspect of this invention also is to provide an improved process for the synthesis of crotyl and secondary-butenyl derivatives (i.e. esters or ethers). A further object is to provide a process for the synthesis of these derivatives in higher selectivities. Accordingly, the present invention is an improved process for making crotyl and secondary-butenyl esters or ethers from butadiene comprising:

a. reacting butadiene or a hydrocarbon fraction comprising butadiene with a compound Q having the general formula R¹(CO)ₙ—OH wherein n=0 or 1 and R¹ is a C₂-C₂₀ alkyl or alkenyl group which may be unsubstituted or independently substituted by 1 or 2 C₁-C₂₀ alkoxy groups or by 1 or 2 hydroxy groups, or R¹ is a C₆-C₁₀ aryl group or a C₇-C₁₁ aralkyl group or a methyl group, with the proviso that R¹ contains no hydroxy substituent if n=1, in the presence of an acid such as a Brönsted acid to form a mixture comprising at least (i) the crotyl derivative and (ii) the secondary-butenyl derivative of the compound Q,

b. subjecting at least part of the reaction mixture to a separation step to remove at least a part of the crotyl derivative (i) and/or the secondary butenyl derivative (ii) from the reaction mixture,

c. recycling to the first stage (a) of the process at least a portion of the reaction mixture from which the derivative (i) and/or (ii) has been removed, said portion comprising at least one or more by-products derived from (iii) butadiene dimerisation or (iv) oligomerisation or (v) reaction of such dimerisation or oligomerisation with compound Q, and

d. recovering the crotyl and/or secondary butenyl derivatives separated in step (b).

In another aspect, the invention provides a process for producing at least one product stream of selected composition, comprising:

(a) combining a hydrocarbon stream comprising a C₄-C₁₀ conjugated diene with a compound Q selected from compounds defined as:
R¹(CO)ₙ—OH
wherein n is 1 or 0, and
R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups,
   under addition reaction conditions to form a reaction mixture containing at least one allyl addition product;

(b) separating the reaction product mixture into streams comprising at least one allyl product stream, and at least one stream containing other reacted and unreacted products;

(c) maintaining a reaction mixture, wherein components of at least at least a portion of at least one of said separated streams from step (a) is subjected to reaction conditions under which (i) C₄-C₁₀ conjugated diene, (ii) compound Q and (iii) allyl addition product participate in an equilibrium reaction
(i)+(ii) [symbol] (iii);

(d) controlling the amount of components (i), (ii) and (iii) in said reaction mixture by adjusting the size of said portion of at least one of said separated streams that is subjected to the reaction conditions of step (c);

(e) recovering at least one component of the separated streams from step (a) and of the reaction mixture of step (c); and

(f) optionally, subjecting one or more recovered components to one or more process selected from hydrolysis, hydrogenation, isomerization, and cracking to form product derivatives in preselected proportions.

By way of example, the present process may be readily adapted to the reaction of butadiene with, for example, acetic acid, to form a mixture of the esters n-but-2-enyl acetate (also known as crotyl acetate, a C₄ acetate) and secondary but-2-enyl acetate (a C₄ acetate), the desired C₄ acetate(s) preferably being separated from one another. If only one of the said C₄ esters is the target product, the other C₄ ester can be recycled to the initial reaction stage (a). Such recycling can be with or without separation from the reaction by-products.

Similarly, the process can be readily adapted to the reaction of butadiene with mono- or dihydric alcohols to produce the corresponding ethers or glycol ethers. In this case the main products of, for example, the reaction of an alkanol with the butadiene are the crotyl ether (1-alkoxy-but-2-ene) and secondary butenyl ether (3-alkoxy-but-1-ene). These two isomers can be separated and isolated or recycled to the reaction stage as indicated for the analogous acetate esters referred to above.

When the target product is the crotyl derivative of compound Q rather than its isomer, the secondary butenyl derivative, said secondary butenyl derivative can be recycled directly or indirectly to Stage (a) of the process if desired. Direct recycle to Stage (a) is believed to result in the isomerisation of the secondary butenyl derivative into at least some crotyl derivative in accordance with the chemical dynamic equilibria existing in the reaction mixture. Similarly, when the target product is the secondary butenyl rather than the isomeric crotyl derivative, the crotyl derivative can be recycled to the Stage 1 reaction. Another option under the above circumstances is to crack the unwanted isomer back to the starting materials, butadiene and compound Q, and to return one or more of these starting materials to the stage (a) reaction. Processes for the cracking of butenyl esters or ethers to provide butadiene and alcohol or carboxylic acid starting material is well known in the art.

The recycle of the excess feedstock, unwanted isomeric C₄ derivative and reaction by-products can be done in several ways. Two possible ways are (i) the separation by distillation from the target isomer and recycle of other fractions to the addition reactor and (ii) as (i) but with a separate treatment reactor. In the first case (i) process, following recovery of butadiene from the reaction product two columns are suitably provided to allow separation of the isomeric butyl derivatives, i.e. the crotyl derivative and secondary butenyl derivative (step (b)) and to allow for the low levels of water employed giving rise to azeotroping mixtures which can hinder the separation of these isomeric derivatives. If one of the isomeric derivatives is not a target product, this isomer together with excess reactants and by-products can be recovered and recycled to the initial addition reaction. The C₄ derivatives (i.e. the butenyl derivatives) and the majority of the reaction by-products under reaction conditions interconvert with butadiene, free carboxylic acid, and the crotyl derivative. In the second case (ii), pretreatment stages are included in the recycle loop for the conversion of the unwanted C₄ derivative and/or reaction by-products to free carboxylic acid or alcohol and butadiene. This can be conveniently achieved by treatment in the vapour/liquid phase with a acidic support such as an acidic zeolite, and alumina. The use of such a separate pre-treatment prior to the return to the addition reactor (step a) may be beneficial on reaction rate and selectivity grounds. A small proportion of the reaction by-products show no evidence of an existence of a dynamic equilibrium between them and the other reaction products. These materials if not removed could build up in the recycle streams and hence will necessitate a bleed stream from one or more of the recycle loops.

In this process, compound Q has the general formula R¹(CO)ₙ—OH in which n is 1 or 0 and R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups. When n is one, compound Q is a carboxylic acid compound, R¹—COOH, and preferably is a saturated aliphatic carboxylic acid. Preferably, Q does not contain a free hydroxyl if Q is a carboxylic acid (i.e., if n=1) to prevent cross esterification. Preferably, such saturated aliphatic carboxylic acids used in the present invention contain 1-6 carbon atoms. Most preferably, if n is one, compound Q is acetic acid.

However, if n is zero, the compound R¹(CO)ₙ—OH is an alcohol, R¹OH, wherein R¹ is as defined above. When n is zero, Q is preferably a saturated aliphatic alcohol or diol. Preferably, the alcohol, R¹OH, is a saturated C₁ to C₂₀ monohydric alcohol or a C₂ to C20 dihydric alcohol. The alcohol preferably contains up to 10 carbon atoms and more preferably contains up to six carbon atoms. Examples of suitable monohydric alcohols are methanol, ethanol, propanol, isopropanol, n-butanol, sec-butanol, tert-butanol, n-pentanol (amyl alcohol), n-hexanol, benzyl alcohol, n-octanol, and 2-ethylhexanol. Preferably at least one of the hydroxyl groups is primary. A suitable dihydric alcohol may have hydroxyl groups on adjacent carbon atoms or on separated carbon atoms. Examples of suitable dihydric alcohols are ethylene glycol, 1,2-propylene glycol, 1,3-propylene glycol, butane-1,4-diol, hexane-1,4-diol.

In the process of the present invention, when the alcohol is a dihydric alcohol reactant, it is can be, for example, a saturated, aliphatic, straight chain glycol preferably having 2-10 carbon atoms. Ethylene glycol is a preferred dihydric alcohol.

Q is a glycol if n is zero and R¹ is substituted with hydroxy or alkyl hydroxy ether groups. The term “glycol” used in this application includes glycol ether compounds and is represented by:
HO(CHR′CHR″O)ₙH
wherein R′ and R″ are each independently a hydrocarbyl group or, preferably, hydrogen, and n is at least 1, preferably, 1 to 10, and more preferably, 1, 2 or 3.

Suitable hydrocarbyl groups include alkyl groups, for example, those having 1 to 10 carbon atoms. Such alkyl groups may be linear or branched. Preferred alkyl groups are C₁ to C₄ alkyls such as methyl, ethyl, propyl and butyl. In a preferred embodiment, the glycol is either monoethylene glycol (MEG) or diethylene glycol (DEG).

The main reactions expected to occur in the reaction of butadiene with the defined compound Q can be represented diagrammatically by the following scheme: 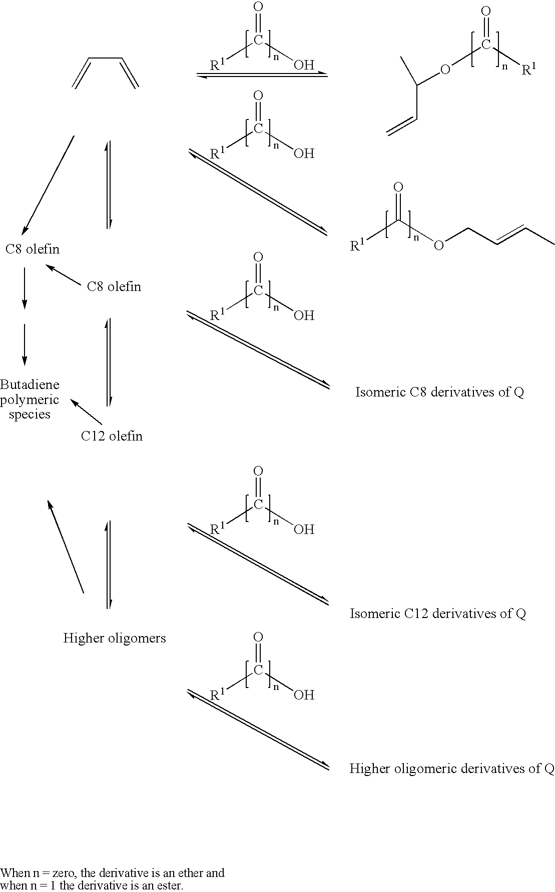
The majority of the reaction products and by-products are formed by reactions which are under equilibrium control. The path on the left side of the diagram (above) showing single arrows represents a possible minor reaction path giving rise to by-products whose recycle does not improve the reaction selectivity.

The build-up of such products can be prevented by separating and discarding a small stream of product from Which useful products and valuable starting materials have been removed. This can be achieved, for example, by having a bleed from a simple recycle loop.

When the major target product is butyraldehyde, the process is controlled to provide a major proportion of the crotyl derivative of compound Q because this is readily converted into butyraldehyde. Under these circumstances, at least some of the secondary butenyl isomer is recycled to the addition reactor where it can isomerise to the crotyl derivative within the dynamic reaction conditions in said reactor.

Similarly, if the major target product is MEK, the process is controlled to provide a major proportion of the secondary butenyl derivative which is readily converted to MEK. Under these circumstances, at least some of the crotyl isomer is recycled to the addition reactor where it can isomerise to the secondary butenyl derivative.

Another option under the above circumstances is to crack some of the isomer in the lesser amount back to the starting materials, butadiene, and compound Q, and to return one or more of these starting materials to the stage (a) reaction. Processes for the cracking of butenyl esters or ethers to provide butadiene and alcohol or carboxylic acid starting material is well known in the art.

Thus in one embodiment of the present invention, butadiene is reacted with, for example, acetic acid, to form a mixture of the esters n-but-2-enyl acetate (also known as crotyl acetate) and secondary but-2-enyl acetate. The isomers are at least partly separated from the reaction mixture and at least some of each isomer is converted to provide MEK and butyraldehyde in the desired quantities. The balance of the isomer not required for conversion can be recycled to the addition reactor.

Similarly, the process can be readily adapted to the reaction of butadiene with alcohols to produce the corresponding ethers. In this case the main products of, for example, the reaction of an alkanol with the butadiene are the crotyl ether (1-alkoxy-but-2-ene) and secondary butenyl ether (3-alkoxy-but-1-ene). These two isomers can be separated and isolated or recycled to the reaction stage as indicated for the analogous acetate esters referred to above.

The reaction between the butadiene and the compound Q is suitably carried out in the liquid phase in the presence of a solvent. It is not essential that both the reactants dissolve completely in the solvent. However, it is an advantage if the solvent chosen is such that it is suitably capable of dissolving both the reactants. Specific examples of such solvents include hydrocarbons such as e.g. decane and toluene and oxygenated solvents such as butyl acetate or excess carboxylic acid (for Q=carboxylic acid) and excess alcohol (for Q=alcohol) reactant. The use of an excess of the compound Q as a reactant can be advantageous when the process of the present invention is used to extract butadiene from an impure stream as it facilitates reaction at high conversion of butadiene or in process terms high efficiency of removal of butadiene. Currently the removal or recovery of butadiene from refinery streams requires a separate processing stage.

The reaction in step (a) in the process of the present invention can be conducted in a homogeneous or heterogeneous phase. Although if a catalyst is used, the process is suitably conducted in the heterogeneous phase for ease of separation of the products from the reaction mixture. The heterogeneous catalyst phase can be liquid strong acids (e.g. acidic ionic liquids, liquid acidic polymers, and partially solvated polymers) or solid strong acids (e.g. HY zeolite, strong acid macrorecticular and gel type ion-exchange resins, and heteropolyacids of tungsten or molybdenum, which have been ion-exchanged and/or supported on a carrier material). Where a homogeneous catalyst is employed it is dissolved in the reaction mixture and can be a strong acid such as sulphonic acid (mono-, di- and poly-sulphonic acid) or a heteropolyacid. A strong acid is defined as an acid having a pKa of one or less.

A preferable process of this invention uses soluble homogenous catalysts. Suitable examples of catalysts that may be used include, sulphonic acids, sulphonic acid substituted polymers such as strong acid ion-exchange resins e.g. amberlyst 15H, phosphoric acid functionalised polymers, acidic oxides e.g. HY zeolites, strong Lewis acids e.g. lanthanide triflate salts, organic sulphonic acids such as methane sulphonic acid, orgainc di- and tri-sulphonic acids, sulphonated calixarenes, heteropolyacids such as tungsten Keggin structure, strong acid ionic liquids such as those described in our prior published EP-A-693088, WO 95/21872 and EP-A-558187. The activity of the above catalysts can be further modified by the use of additives such as alkyl pyridinium, quaternary alkyl ammonium and quaternary phosphonium compounds each of which may be the halides, sulphates or carboxylates. In addition to these, the presence of water as a reaction adjuvant can also beneficially affect the activity and selectivity of the catalysts. In the process of the present invention it is also advantageous to use polymerisation inhibitors such as e.g. alkylated phenols such as BHT butylated hydroxytoluene also called 2,6-di-tert-butyl-p-cresol, other members of this series include the Irganox series of materials from Ciba Specialty Chemicals, Lowinox series of materials from Great Lakes Chemical Corporation, tropanol series from ICI, and t-butylcatechol, nitroxides such as nitoxides and nitroxide precursors di-t-butylnitroxide, and n,n-dimethyl4-nitrosoaniline, nitric oxide, stable radicals such as 2,2,6,6,-tetramethyl-piperidine-1-oxyl, 2,2,6,6,-tetramethyl4-hydroxypiperidine-1-oxyl and 2,2,6,6,tetramethylpyrrolidine-1-oxyl, to prevent the polymerisation/oligomerisation of the butadiene reactant into unwanted polymers in the presence of the aforementioned acidic catalysts.

Other examples of catalysts suitable for use in the addition reaction of the butadiene to compound Q are heterogeneous catalysts based on strong acid macorecticular ion-exchange resins with a proportion of the acidic sites exchanged with bulky counterions such as e.g. a bi-carbonium counter ion.

Typically these counterions account for less than 10% of the available acidic sites. It has been found that low levels of water are required, at levels above 5% w/w the catalyst activity is significantly reduced whereas at levels below 0.05% w/w, the activity though high is rapidly lost due to deactivation of the catalyst. Consequently the water level in the reaction zone is suitably in the range from 0.05 to 5% w/w on the carboxylic acid, preferably from 0.05 to 1% w/w.

A sulphonic acid catalyst may be used in the process of the present invention, especially in addition of butadiene to an alcohol or glycol, typically in a ratio of the number of carbon atoms to sulphonic acid groups is preferably in the range 1:1 to 1:0.2, more preferably in the range 1:1 to 1:0.5 and most preferably in the range 1:1 to 1:0.7. The sulphonic acid preferably contains 2 to 30 carbon atoms, more preferably 2 to 10 carbon atoms and most preferably 2 to 8 carbon atoms. Examples of suitable sulphonic acid catalysts are 1,2-ethane disulphonic acid, benzene-1,2-disulphonic acid, benzene-1,3-disulphonic acid, benzene-1,4-disulphonic acid, naphthalene-1,5-disulphonic acid, naphthalene-2,6-disulphonic acid, naphthalene-2,7-disulphonic acid, 4-chlorobenzene-1,3-disulphonic acid, 4-fluorobenzene-1,3-disulphonic acid, 4-bromobenzene-1,3-disulphonic acid, 4,6-dichlorobenzene-1,3-disulphonic acid, 2,5-dichlorobenzene-1,3-disulphonic acid, 2,4,6-trichlorobenzene-1,3-disulphonic acid, 3-chloronaphthalene-2,6-disulphonic acid, benzene trisulphonic acid and naphthalene trisulphonic acid.

The sulphonic acid catalyst employed in the present invention contains at least two sulphonic acid groups per molecule. The sulphonic acid catalyst can comprise a single sulphonic acid compound or a plurality of different sulphonic acid compounds provided that the overall average carbon: sulphonic acid ratio for the catalyst is in the range 1:1 to 1:0.15 and that at least 50 wt % of the component sulphonic acid compounds contain at least 2 sulphonic acid groups per molecule.

The concentration of sulphonic acid catalyst employed in the liquid phase of the reaction mixture can be maintained constant throughout the reaction, or can be varied or can be allowed to vary within a broad concentration range whilst still achieving desirable results. The reaction can be carried out, for example, under batch or continuous conditions.

Under batch conditions, preferably, a single aliquot of the sulphonic acid catalyst is dissolved in one of the reactants, preferably the alcohol, and to continuously or intermittently add the other reactant thereto. For example, in the reaction of butadiene with ethylene glycol, the sulphonic acid can be dissolved in the glycol and the butadiene (in gaseous or liquid form) can be gradually pumped into the reaction mixture. Under these conditions, the concentration of catalyst generally decreases due to the dilution effect as more and more diene enters the liquid phase with the formation of liquid ether. Another method of carrying out the reaction is to continuously or intermittently feed the diene and or catalyst and/or alcohol to maintain the concentrations of catalyst and reactants at the desired level. The catalyst can be fed in as solid or as a liquid. The catalyst fed to the reactor can be dissolved in solvent or in one of the reactants if desired, e.g. the catalyst can be dissolved in additional alcohol or diene reactant if desired.

Preferably a sulphonic acid catalyst concentration is maintained in the range 0.2 to 10 weight %, preferably 0.5 to 7 wt %, most preferably 1 to 5 wt % based on the eight of the sulphonic acid catalyst in the total reaction mixture. The sulphonic acid catalysts of the present invention are preferably soluble in the reaction mixture. Typically, the reaction mixture forms a single liquid phase, but may comprise two or more phases.

Fouling does not deactivate homogeneous catalysts, i.e. catalysts that are soluble in the reaction mixture, and accordingly, use of such catalysts in the present invention largely overcomes the fouling problems associated with the use of heterogeneous catalyst systems employed in some prior art methods. The reaction between the alcohol and the diene is preferably carried out in the presence of water. For example, the liquid phase can contain 0.01 to 10 wt %, and more preferably 0.05 to 4 wt % water based on the total liquid phase.

The reaction is suitably carried out in the liquid or mixed liquid/gas phase in the presence of a solvent. The reaction is preferably carried out under conditions such that the reaction between the diene and the alcohol occurs in the liquid phase. If a solvent is employed, it is not essential that both reactants dissolve completely in the solvent. However, it is an advantage if the solvent chosen is such that it is capable of dissolving both the reactants and the catalyst. Specific examples of such solvents include hydrocarbons such as decane and toluene and oxygenated solvents such as glymes and ethers, for example, 1,2,-dibutoxyethane, tetrahydrofuran and 1,4-dioxane.

A further feature of the present invention provides for the separation of the sulphonic acid catalyst from any involatile residues that may build up in the reaction mixture, and the recycle of this recovered catalyst to the reactor. Such separation can be achieved by virtue of the fact that the sulphonic acids are generally soluble in water, whereas the residues are either insoluble, or soluble only in the organic components of the reaction mixture. Thus, for example recovery of the sulphonic acids can be achieved by treatment of the reaction mixture with water, or by treating the involatile residues from the reaction mixture with water and thereafter separating the aqueous phase from any residue. The aqueous phase is preferably extracted with an immiscible organic solvent, for example cyclohexane, to assist the purification of the aqueous sulphonic acid solution. The aqueous phase containing the sulphonic acid catalyst can then be concentrated if necessary and returned to the reactor.

Alternatively, the separation of the catalyst, for recycle, from the reaction mixture can be achieved simply by decantation in the case of a heterogeneous phase liquid catalyst system. This maybe facilitated by cooling or adding water to facilitate the phase separation. In the case of a fully homogeneous catalyst the volatile reaction products can be separated by either flash distillation or falling film evaporation. Key features of this are optimisation to reduce further reaction or back reaction, varying residence time, temperature and pressure. Low values of each of these will facilitate this.

Relative mole ratios of butadiene to the compound Q in the addition reaction is suitably in the range from 5:1 to 1:50, preferably in the range from 1:1 to 1:10.

If compound Q is a carboxylic acid, the addition reaction (step (a)) is suitably carried out at a temperature in the range from 20 to 150° C., preferably from 40 to 110° C. However, if compound Q is a monohydric or dihydric alcohol, the addition reaction (step (a)) is suitably carried out at a temperature of above 20, preferably above 40 and typically above 60° C. and the temperature may range up to 130, preferably up to 120, and typically up to 110° C. A preferable range is 40 to 90° C.

The reaction can be carried out at any desired pressure, but is preferably carried out at the autogenous reaction pressure, which is determined by factors such as the reaction temperature, presence or absence of solvent, excess of reactants and impurities present in the butadiene stream. An additional pressure may be applied to the system if a single fluid phase is preferred e.g. there is no butadiene gas phase in addition to the solvated liquid phase. In the case of impure butadiene streams such as crude C₄'s, the gas phase may consist of other components, which can add to the total system pressure.

Apparatus for carrying out an addition reaction is well known to those skilled in the art. The addition reaction (step (a)) may be suitably carried out in a plug flow reactor, with the unused butadiene being flashed off and recycled to the reactor via a vapour liquid separator, but equally could be conducted in a slurry reactor. In the case of a plug flow reactor, the butadiene can be present partially as a separate gas phase as well as being dissolved and this would result in either a trickle bed operation or a bubble bed operation. Diene feed can be added for example at a plurality of places in the reactor (e.g. at intervals along the length of a plug flow reactor). In the case of a bubble bed device, the diene can, if desired, be added counter-current to the reactant feed. A typical LHSV (liquid hourly space velocity=volume of liquid feed /catalyst bed volume) for the compound Q is 0.5 to 20 more preferably 1 to 5. In the case of a slurry reactor, a continuous bleed of any deactivated catalyst can be taken. It is economically advantageous to run with catalyst in a various stages of deactivation to improve the utilisation of catalyst. In this case the total loading of catalyst (activated+deactivated) can reach high levels such as 50% w/w of the reaction charge. The butadiene feed can be added sequentially along the length of the reactor and with the Q feed or in a counterflow mode. Preferably, diene may be added gradually to a reactant such as an alcohol, for example, by multiple injections at constant pressure in a batch reactor. By adding the diene gradually in this manner, side reactions such as diene polymerisation can be minimised.

Isomerisation of allyl (e.g., crotyl and sec-butenyl) derivatives to corresponding enol derivatives can be catalysed by either strong non-nucleophilic bases or transition metal complexes. A strong base catalytic material may be heterogeneous or homogeneous. If the derivative contains a carboxylate functionality, the mixture to be treated should be substantially free of free carboxylic acid and water otherwise catalyst deactivation can result from neutralisation and saponification. For alcohol based derivatives strong bases such as sodium methoxide and potassium t-butoxide are suitable (J. Amer. Chem. Soc 111 6666 (1989), J. Org. Chem 53 1860 (1988), J. Chem. Soc Perkin I 1535 (1972) 1858(1973)).

Isomerisation of crotyl and sec-butenyl derivatives to the corresponding enol derivatives also may be carried out using transition metal complexes. These reactions may be homogeneous or heterogeneous.

The isomerisation can be carried out, in the gaseous phase or in the liquid phase. When carrying out these reactions in the liquid phase either homogeneous or heterogeneous catalysts can be used. If these process stages are operated in the gaseous phase, heterogeneous catalysts are preferred in general. The homogeneous catalysts used for the isomerisation can be selected from a variety of transition metal element compounds, particularly those containing Groups 1, 5, 6, 7, 8, 9, and 10 (formerly known as Groups Ib, Vb, VIb, VIIb, and VIIIb) elements, preferably copper, vanadium, chromium, molybdenum, tungsten, rhenium, iron, cobalt, nickel, ruthenium, rhodium, palladium, platinum, osmium and/or iridium. Suitable catalysts are, for example, the salts of these transition metals, particularly their halides, nitrates, sulphates, phosphates, or carboxylates soluble in the reaction medium, for example, their C₁-C₂₀ carboxylates, such as formates, acetates, propionates, 2-ethylhexanoates, and also the citrates, tartrates, malates, malonates, maleates, or fumarates, sulfonates, for example, methanesulfonates, benzenesulfonates, naphthalenesulfonates, toluenesulfonates, or trifluoromethanesulfonates, cyanides, tetrafluoroborates, perchlorates, or hexafluorophosphates, also soluble salts of the oxy-acids of these metals, particularly the alkali metal, alkaline earth metal, or onium salts, such as ammonium, phosphonium, arsonium, or stibonium salts, of vanadium oxy-acids, rhenium oxy-acids, or perrhenic acid, or the anhydrides of these acids, particularly dirhenium heptoxide, soluble inorganic complex compounds of these elements, particularly their aquo, ammine, halo, phosphine, phosphite, cyano, or amino complexes as well as the complexes of these transition metals with chelating agents such as acetylacetone, dioximes, for example, diacetyldioxime, furildioxime, or benzildioxime, ethylenediaminetetraacetic acid, nitrilotriacetic acid, nitrilotriethanol, ureas or thioureas, bisphosphines, bisphosphites, bipyridines, terpyridines, phenanthrolines, 8-hydroxyquinoline, crown ethers or poly(alkylene glycol)s, as well as organometallic compounds of these transition metal elements, for example, carbonyl complexes such as HRuCl(CO)(PPh₃)₃, HRuCl(CO)(hexyldiphenylphosphine)₃, RuH₂ (CO)(PPh₃)₃, RuH₂ (PPh)₃ or IrCl(CO)(PPh₃)₃ (the abbreviation PPh3 designating triphenylphosphine); Fe₂ (CO)₉ or Fe₃ (CO)₁₂; or organotrioxorhenium(VII) compounds such as C₁-C₄ alkyltrioxorhenium(VII), particularly methyltrioxorhenium(VII), cyclopentadienyl trioxorhenium(VII), or phenyltrioxorhenium(VII).

Examples of supports that may be used for such catalysts in order to render them heterogeneous with respect to the isomerisation step include supports containing acidic residues, for example strongly acidic ion exchange resins, and sulphonic acids such as paratoluenesulphonic acid. It is possible to carry out this process stage using a heterogeneous catalyst.

The supported strong base catalysts referred to above enable a process with a sequential or simultaneous/combined isomerisation and hydrolysis stages to be employed. It is essential for carboxylic acid derivatives in this case that the isomerisation stage either precedes the hydrolysis stage or is carried out simultaneously with the hydrolysis stage. Otherwise, the ester will split into an allylic alcohol in equilibrium with the carboxylic acid and it will be necessary to devise methods of shifting the equilibrium in the desired direction. The recovery of the lower boiling point n-butyraldehyde and methyl ethyl ketone from Q is likely to be more energy efficient than separating the derivatives. The butyraldehyde and methyl ethyl ketone so formed may be optionally converted to derivatives, for example, catalytically hydrogenated to form n-butanol and sec-butanol.

The isomerisation and hydrolysis stages also may be conducted consecutively or sequentially. It is possible to combine isomerisation of the crotyl and sec-butenyl derivatives to enol esters (or ethers) and hydrolysis to give mixtures of butyraldehyde and methyl ethyl ketone.

Unsaturated products of this invention may be converted to corresponding saturated derivatives by hydrogenation. Hydrogenation may be carried out under heterogeneous conditions over any suitable catalyst. Examples of suitable catalysts include ruthenium, platinum, nickel (e.g., Raney Ni) and palladium, which may be employed as metals or metal compounds. Although unsupported catalysts may be employed, it is preferable to use catalysts supported on inert carriers, such as carbon or siliceous supports. Preferred catalysts include supported Raney nickels, and ruthenium on carbon.

Some reactants, such as a glycol, from a previous reaction stage may be present and this may have a detrimental effect on some catalysts. A solvent is not required for this reaction. The reaction can be carried out in an all gas/vapour phase or as a two-phase mixture. In the latter case a flow reactor would be operated in either a trickle bed or a bubble bed mode (no hydrogen is needed). For example, completion of hydrogenation of n-but-2-enyl glycol ethers can be determined conveniently for batch reactions by cessation of hydrogen uptake and in the case of both flow and batch reactors by sampling and analysis by methods such as Gas Chromatography and ultraviolet (UV) spectroscopy.

The hydrogenation may be carried out at 20 to 200° C., preferably, 40 to 160° C. The hydrogenation may be carried at a pressure of 1 to 100 barg, preferably, 5 to 50 barg. The hydrogenation can be carried out in a slurry and/or flow reactor.

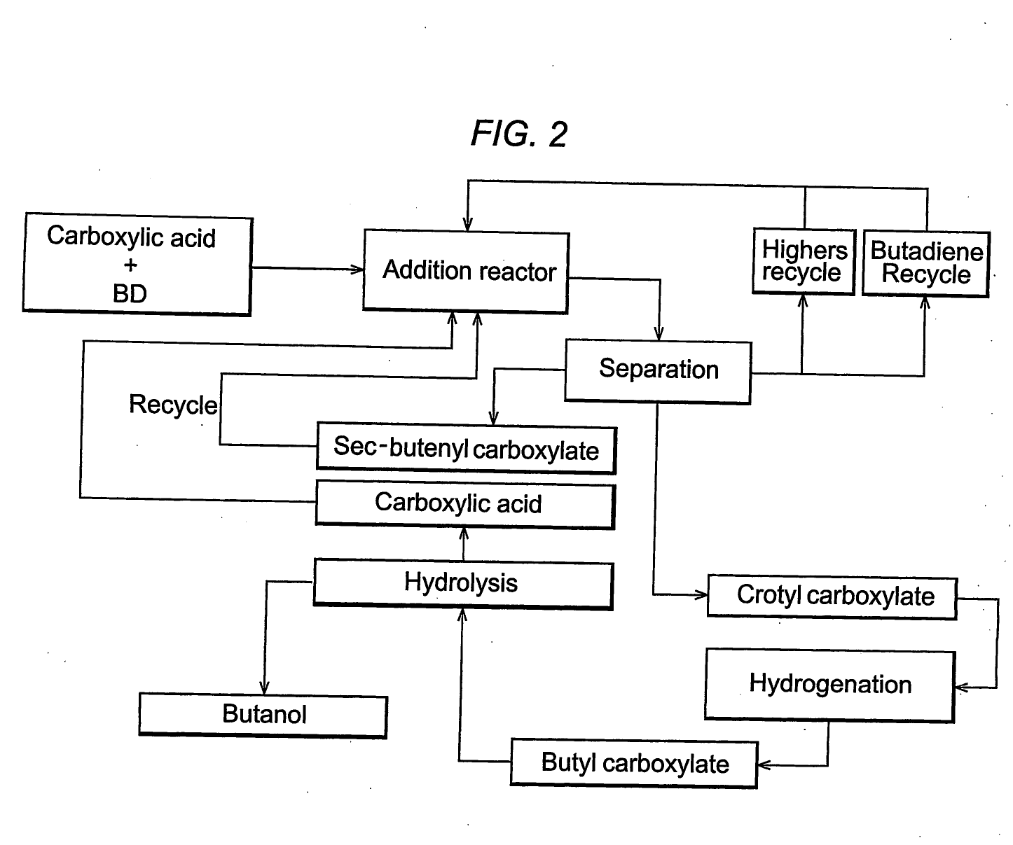

Direct addition of butadiene is illustrated by some examples. FIG. 2 of the Drawings shows a possible scheme for co-production of butanol and the butyl derivative of a carboxylic acid such as acetic acid. When the carboxylic acid is acetic acid this scheme describes a route for the co-production of n-butanol and butyl acetate. The process of the present invention is for improvement of the chemical efficiency of the butadiene addition stage by recovery and recycle of reaction by-products such as C₈ olefins, C₈ carboxylate, C₁₂ olefins, C₁₂ carboxylates and other higher carbon number butadiene and carboxylate containing materials. These are shown in the diagram as a “highers” recycle. This recycle stream is conveniently obtained during the butadiene addition product allylic alcohol recovery stage shown as a purification box. Typically the separation of products is achieved by distillation and the relative order of the boiling points of the reaction products; production of by-products depends on the choice of the starting reactant, e.g., a carboxylic acid. In the case of acetic acid, some of the “highers” can form some water azeotropes but in the absence of water behave as a higher boiling point fraction than acetic acid and the highers can be recovered as a high boiling point stream. Alternatively these materials by virtue of their decreased water solubility compared to acetic acid can be recovered by water addition and decantation of the upper predominately organic layer. In practice the low levels of high boiling point materials that are not in dynamic equilibrium with the starting materials as part of the highers recycle stream could build up to unacceptable levels if a bleed from the recycle is not taken. Water extraction may be used to recover valuable carboxylic acid as an aqueous solution for recycle from this process bleed.

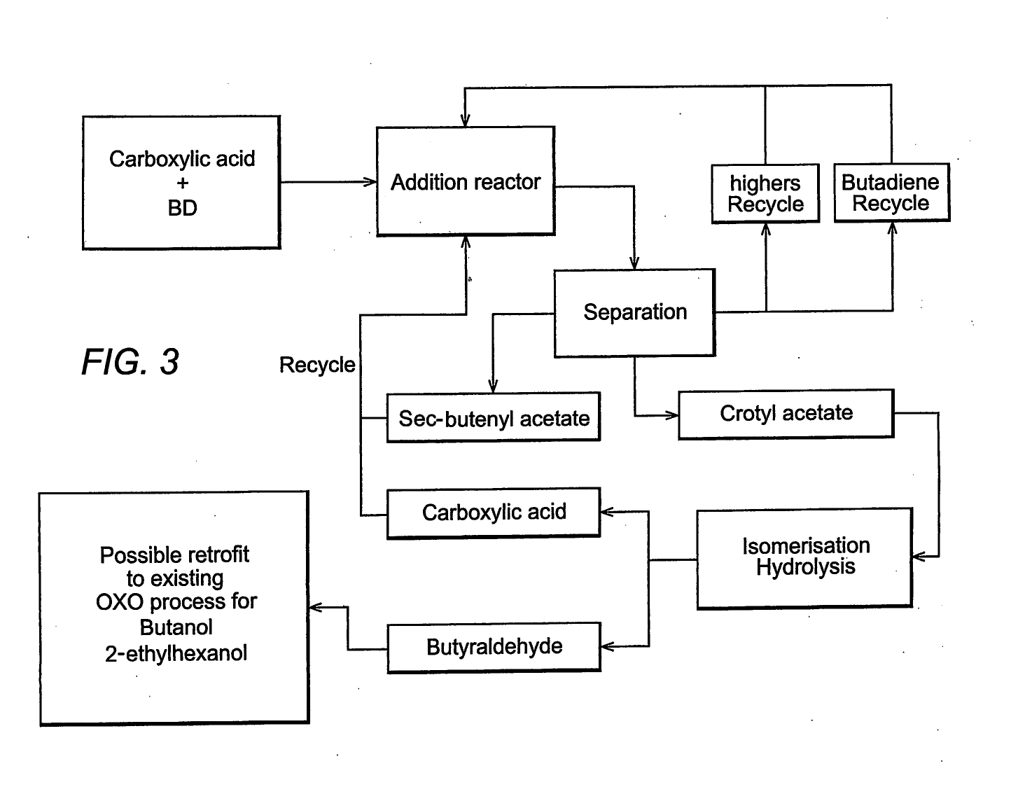

The crotyl carboxylate produced by the reaction of butadiene with a carboxylic acid can also be converted to butyraldehyde by isomerisation and hydrolysis. FIG. 3 shows a possible scheme for co-production of butyraldehyde, butanol and 2-ethylhexanol that can be used to replace the hydroformylation stage of an OXO process to n-butanol and 2-ethylhexanol. The schemes illustrated in FIGS. 2 and 3 have a common intermediate crotyl acetate and can be combined to co-produce butanol, butyl carboxylate, butyraldehyde and 2-ethylhexanol.

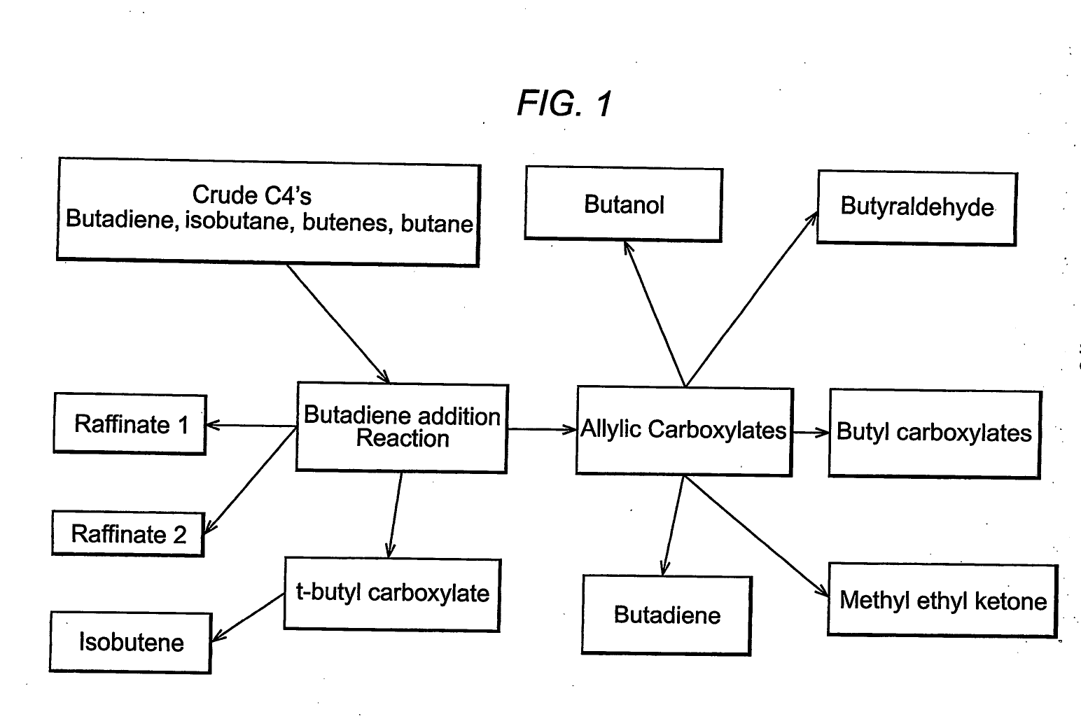

Steam cracking of naphtha produces among other products a raw C₄ stream, which contains butadiene, isobutene, butenes and butanes as major components. This is often selectively hydrogenated to remove trace acetylenic impurities and is referred to as a crude C₄ stream. The refinery crude C₄ stream currently is used in several ways. A process in commercial operation is to extract the butadiene to produce a stream containing predominately isobutene, butenes, butane and trace butadiene, this is commonly selectively hydrogenated to convert the residual butadiene to butenes and the resultant product is referred to as a raffinate 1. Raffinate 1 finds use as a raw material as an alkylation feedstock and for its constituent components. For example, raffinate 1 is often reacted with methanol to produce methyl t-butyl ether, commonly called MTBE, which in turn is eliminated to generate a purified isobutene stream and re-liberate methanol for recycle. The purified isobutene stream is a valuable chemical intermediate and finds use in polyisobutene, and methacrylic acid manufacture. A by-product of this isobutene extraction stage is a stream containing predominately butenes and butane with trace isobutene that is commonly referred to as raffinate 2. The reaction of a crude C₄ stream with carboxylic acid provides an alternative route to extracting valuable olefinic chemicals whilst co-producing valuable oxygenates. For example, both the crotyl and sec-butenyl carboxylates are know to be in equilibrium with free butadiene and carboxylic acid in the presence of a Brönsted acid and as a result the formation and isolation of these allylic carboxylates can be used to extract butadiene and generate a pure butadiene stream from a hydrocarbon stream such as crude C₄. The carboxylic acid under the reaction conditions for butadiene addition is also active towards addition to isobutene and produces an equilibrium-limited amount of t-butyl ester. This reaction itself is reversible under Brönsted acid catalysis and the isolated t-butyl ester can be used as a process intermediate to produce a pure isobutene stream. The removal of butadiene and isobutene from a crude C₄ stream will produce a raffinate 2. Alternatively, t-butyl ester may be recycled to the addition reactor where a standing level of t-butyl ester can be used to suppress the forward reaction of isobutene. This produces raffinate 1 which is a crude C₄ stream minus butadiene (a selective hydrogenation to remove residual butadiene used in refineries also may be required). From the above considerations it can be seen that the addition of carboxylic acids can be used to isolate valuable components from a hydrocarbon stream such as crude C₄ e.g. butadiene, isobutene, raffinate 1, raffinate 2. An important common feature of this chemistry is the reaction of butadiene with carboxylic acids. The process of the present invention by improvement of the chemical efficiency of the butadiene addition stage provides an overall improvement of the refinery integration and oxygenated product options. FIG. 1 shows some of these options.

The above chemistry described for carboxylic acids has parallel chemistry for the alcohol derivatives of the present invention. For example, in the case of methanol the reaction of crude C₄ will result in allylic ethers and MTBE (methyl tertiary butyl ether). The MTBE can be cracked back to isobutene and methanol using well-established refinery chemistry. In the case of the ethylene glycol derivative, this chemistry allows access to the butyl glycol ethers, which are important industrial solvents. The importance of recovery and recycle of reaction by-products for the glycol case is increased as a significant amount of di-substitution by reaction of butadiene with both alcohol functionalities can occur. For example, the reaction of butadiene with ethylene can give rise to three main di-substituted species e.g. 1,2-di (crotoxy) ethane, 1,2-di (sec-butenoxy) ethane, 1-crotoxy-2-sec-butoxy ethane.

Another aspect of the present invention to provide an improved process for the co-production of methyl ethyl ketone (MEK) and butyraldehyde under conditions which permit the ratio of MEK to aldehyde to be controlled at will. It is a further aspect to provide an improved process for the co-production of methyl ethyl ketone (MEK) and butyraldehyde wherein the overall formation of unwanted by-products is reduced.

In one aspect of this invention, conversion of butadiene to methyl ethyl ketone and butyraldehyde is performed by addition of a carboxylic acid or alcohol to butadiene to form a mixture of isomeric allylic esters (or ethers), n-1-but-2-enyl ester (or ether) and a secondary 3-but-1-enyl ester (or ether); partial recycling of one of the allylic esters (or ether)s from said mixture of products, then treatment of the remaining allylic esters (or ethers) to isomerisation and hydrolysis stage to give methyl ethyl ketone and n-butyraldehyde.

Accordingly, the present invention provides a process for co-production in controlled proportions of methyl ethyl ketone and butyraldehyde by

a. reacting butadiene or a hydrocarbon fraction comprising butadiene with a compound Q having the general formula R¹(CO)ₙ—OH wherein n=0 or 1 and R¹ is a C₂-C₂₀ alkyl or alkenyl group which may be unsubstituted or substituted by 1 or 2 C₁-C₂₀ alkoxy groups or R¹ is a C₆-C₁₀ aryl group or a C₇-C₁₁ aralkyl group or a methyl group, in the presence of a Brönsted acid to form a mixture comprising at least (i) a crotyl derivative of compound Q and (ii) a secondary-butenyl derivative of the compound Q,

b. continuously or intermittently subjecting at least part of the reaction mixture to one or more separation processes to (b1) separate at least part of the crotyl derivative and (b2) separate at least part of the secondary-butenyl derivative, steps (b1) and (b2) being carried out simultaneously or sequentially and in any order,

c. recycling to the first stage (a) of the process at least a portion of the reaction mixture from which the crotyl derivative and/or the secondary-butenyl derivative has been removed, said portion comprising at least one or more by-products of the stage (a) reaction, the by-products being derived from (i) butadiene dimerisation, or (ii) butadiene oligomerisation, or (iii) reaction of such dimerisation or oligomerisation products with compound Q, and

d. converting the separated crotyl derivative to butyraldehyde and converting the separated but-2-enyl derivative to methyl ethyl ketone.

A further aspect of the present invention provides a process for co-production in controlled proportions of methyl ethyl ketone and butyraldehyde by

a. reacting butadiene or a hydrocarbon fraction comprising butadiene with a compound Q having the general formula R¹OH wherein R¹ is a C₂-C₂₀ alkyl or alkenyl group which may be unsubstituted or substituted by 1 or 2 C₁-C₂₀ alkoxy groups or R¹ is a C₆-C₁₀ aryl group or a C₇-C₁₁ aralkyl group or a methyl group, in the presence of a Brönsted acid to form a mixture comprising at least (i) a crotyl ether of compound Q and (ii) a secondary-butenyl ether of the compound Q.

b. continuously or intermittently subjecting at least part of the reaction mixture to one or more separation processes to (b1) separate at least part of the crotyl ether and to (b2) separate at least part of the secondary-butenyl ether, steps (b1) and (b2) being carried out simultaneously or sequentially and in any order, the separation being carried out to isolate the desired quantities of crotyl ether: sec-butenyl ether for conversion into methyl ethyl ketone and butyraldehyde,

c. recycling to the first stage (a) of the process, directly or indirectly, at least some crotyl ether or secondary butenyl ether, and

d. converting the desired quantity of the separated crotyl derivative to butyraldehyde and the but-2-enyl derivative to methyl ethyl ketone.

An example of the process of this invention is formation of methyl ethyl ketone and butyraldehyde by the addition of carboxylic acids or alcohols to butadiene.

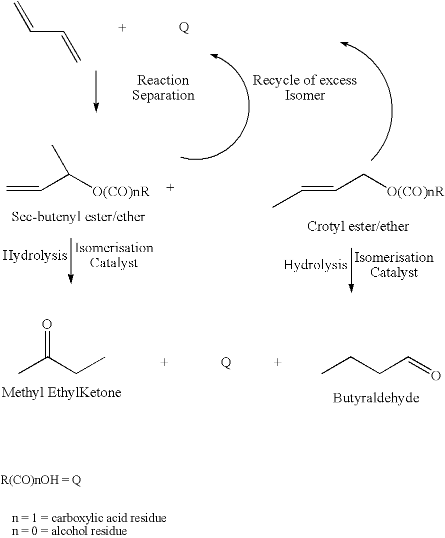

The enol, isomerisation and hydrolysis stages can be carried out in accordance with the methods described in U.S. Pat. No. 6,403,839 (for hydrolysis and isomerisation of the carboxylate derivatives) and U.S. Pat. No. 5,705,707 (for the ether derivatives).

As described in U.S. Pat. No. 6,620,975, mixed C₄ streams may be contacted with a saturated aliphatic glycol in the presence of a catalyst. Under the reaction conditions, butadiene in the C₄ stream reacts with glycol to produce n-butenyl and sec-butenyl glycol ether. The sec-isomer may be recycled to the reactor, or cracked back to the starting materials. The n-isomer, on the other hand, is recovered and hydrogenated to produce n-butyl glycol ether, which is a useful solvent.

Not all the glycol ether initially fed to the reactor is consumed in the butadiene/glycol addition reaction. Instead, some of the glycol ether reacts with the iso-butene present in the mixed C₄ feedstock to produce t-butyl glycol ether. This by-product is isolated from the product mixture and cracked back to iso-butene and glycol ether. The iso-butene is recovered by distillation, and sold, for example, as a feedstock for the production of polyisobutene (PIB). The glycol ether is recycled to the reactor, and may be re-consumed in one of the addition reactions occurring therein.

The cracking and distillation equipment required in the process of U.S. Pat. No. 6,620,975 can add cost and complexity to the overall process. It is therefore among the objects of the present invention to provide an alternative process for treating such mixed C₄ streams.

According to the present invention, there is provided a process for treating a mixed C₄ stream comprising isobutene and 1,3-butadiene, said process comprising:

a) reacting an aliphatic glycol with said stream in an addition reactor,

b) withdrawing a product stream comprising isobutene, sec-butenyl glycol ether, n-butenyl glycol ether and t-butyl glycol ether from the addition reactor, and

c) recovering n-butenyl glycol ether from the product stream, characterised in that

d) t-butyl glycol ether is recycled to said addition reactor.

For the avoidance of doubt, sec-butenyl glycol ether and n-butenyl glycol ether have the following structures: 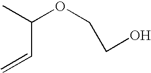
sec-butenyl glycol ether, and 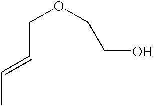
n-butenyl glycol ether.

Under the operating conditions of the addition reactor, t-butyl glycol ether is believed to be in equilibrium predominately with isobutene and glycol, and to a lesser extent, with glymes such as t-butyl glyme (see below). Thus, by recycling the t-butyl glycol ether back to the reactor, the amount of t-butyl glycol ether in the reaction loop is believed eventually to approach a substantially constant value. By controlling the amount of t-butyl glycol ether produced in this manner, the amount of glycol and isobutene consumed in the reaction between isobutene and glycol is maintained at a substantially reduced level. In other words, a significant proportion of the isobutene present in the crude C₄ stream is left unreacted, and consumption of the glycol feedstock is reduced.

The t-butyl glycol ether recycle also is believed to simplify the process, as a separate t-butyl glycol ether cracking stage is no longer required to recover the isobutene. Although isobutene is not produced by cracking t-butyl glycol ether as described in U.S. Pat. No. 6,620,975, any unreacted isobutene originally present in the original mixed C₄ stream may be recovered, for example, by boiling point, as will be described below. The mixed C₄ stream employed as a feedstock in the present process may be a by-product of a reaction, such as butane or butene dehydrogenation or naphtha steam cracking. Such mixed C₄ streams may comprise isobutene and 1,3-butadiene. The mixed C₄ stream may also comprise one or more of isobutane, n-butane, 1-butene, trans-2-butene, cis-2-butene, 1,2-butadiene, propadiene, methyl acetylene, ethyl acetylene, dimethyl acetylene, vinyl acetylene, diacetylene, and C₅ acetylenes. In one embodiment, the mixed C₄ stream is a by-product of naphtha steam cracking comprising isobutane (e.g., 1-2 % v/v), n-butane (e.g., 2-4% v/v), isobutene (e.g., 25-29% v/v), 1-butene (e.g., 8-10% v/v), trans-2-butene (e.g., 6-8% v/v), cis-2-butene (e.g., 3-5% v/v), 1,3-butadiene (e.g., 43-48% v/v), 1 ,2-butadiene (e.g., 0-2% v/v), propadiene (e.g., 0-1% v/v), methyl acetylene (e.g., 0-1% v/v), ethyl acetylene (e.g., 0-1% v/v), dimethyl acetylene (e.g., 0-1% v/v), vinyl acetylene (e.g., 0-1% v/v), diacetylene (e.g., 0-trace) and C₅ acetylenes (e.g., 0-trace). The precise composition of the latter stream may vary depending on factors such as the naphtha feed composition and the how the cracker is operated.

As described in step a), the mixed C₄ stream is reacted with glycol in an addition reactor. The reaction conditions employed in the addition step are described in detail in U.S. Pat. No. 6,620,975. The relative mole ratios of butadiene in the mixed C₄ stream to glycol ether may be 5:1 to 1:50, preferably, 1:1 to 1:10.

Preferably, the addition reaction may be carried out at a temperature of 20 to 170° C., preferably, 50 to 150° C., and more preferably, 70 to 120° C. The reaction may be carried out using a homogeneous or heterogeneous catalyst as described above.

Water may be present in the addition step typically in an amount between 0.01 and 5, preferably, 0.05 and 2 wt %, based on the total charge to the reactor. Although higher amounts of water could be present, activity and selectivity will decrease.

In certain cases, the activity of heterogeneous catalysts may decrease after prolonged use. This may be due to blockage of active sites by butadiene oligo- and polymerisation products. In such cases, it may be advantageous to carry out the addition reaction under conditions of high shear, as high shear rates are believed to reduce blockage of active sites by the formation of such oligo- and polymerisation products. Alternatively or additionally, polymerisation inhibitors may be added to the reaction mixture. Such inhibitors are well-known in the art. Where oligo- and polymerisation products are present in the product stream, however, these may be recovered and recycled to the reactor.

The addition reaction may be carried out using any suitable reactor. For example, a fixed bed, slurry, trickle bed, bus loop, or fluidised bed reactor may be employed.

The reaction between the mixed C₄ stream and ethylene glycol produces a product stream, which is withdrawn from the addition reactor in step b). This product stream comprises addition products, such n-butenyl glycol ether, sec-butenyl glycol ether, t-butyl glycol ether. Preferably, such addition products account for 1 to 99% w/w, for example, 5 to 50% w/w of the product stream. The n-butenyl and sec-butenyl glycol ethers result from the addition of glycol to 1,3-butadiene, while the t-butyl glycol ether results from the reaction between glycol and iso-butene. Such addition reactions, however, do not generally go to completion and are controlled by a number of factors including how the reaction is conducted (e.g. LHSV), reaction kinetics and equilibrium constants. For this reason, unreacted isobutene and, optionally, unreacted 1,3-butadiene are also present in the product stream. These unreacted C₄ components are relatively volatile, and may be separated, for example, by gas disengagement using any suitable separation unit, such as a flash drum. During such a separation step, other volatile C₄ components in the product stream, such as unreacted isomeric butanes, 1-butene and 2-butene may also be separated. Where butadiene is present in the separated mixture, the separated mixture may be selectively hydrogenated. This selective hydrogenation step predominately converts butadiene to 1-butene. Additionally, some isomerisation to 2-butene can occur, as well as further hydrogenation to butane.

The separated mixture of unreacted C₄ components may be used as a feedstock, for example, for alkylation, or a steam cracker. Alternatively, the mixture of unreacted C₄ components may be separated (eg by physical and/or chemical methods) into one or more components for sale or use. Iso-butene, for example, may be recovered and polymerised to produce polyisobutene (PIB). 1-Butene and/or 2-butene may be separated, for example, as a mixture and used as a fuel additive.

In step c), n-butenyl glycol ether is recovered from the product stream. This may be carried out using any suitable separating unit, for example, one or more distillation columns. Once recovered, the n-butenyl glycol ether may be cracked back to butadiene and glycol ether, or recycled to the reactor. Where a cracking step is used, the butadiene and/or glycol ether produced may be recycled to the reactor. Alternatively, at least one of the components may be put to an alternative use. For example, any butadiene produced in this manner may be used as a feedstock for other chemical reactions, such as the production of a butyl ester from the reaction with carboxylic acid, like acetic acid. The reaction between butadiene and a carboxylic acid to produce butyl esters is described in detail in WO 00/26175 (U.S. Pat. No. 6,465,683). Preferably, however, the recovered n-butenyl glycol ether is hydrogenated as described above.

As described in step d), t-butyl glycol ether is recovered from the product stream and recycled to the reactor. The recovery step may be carried out using any suitable separating unit, for example, a distillation column. The recovered t-butyl glycol ether stream may also comprise other reaction products and/or unreacted reactants including, for example, water and unreacted C₄ compounds.

Optionally, sec-butenyl glycol ether may be recovered from the product stream. The separated sec-butenyl glycol ether may be recycled to the reactor, or isolated for, for example, sale, direct use (such as a solvent), or further processing. In one embodiment of the invention, the sec-butenyl glycol ether is thermally cracked back to butadiene and glycol ether. One or both of these starting materials may be recycled to the reactor. It is also possible to put at least one of the components to an alternative use. For example, any 1,3-butadiene produced in this manner may be used as a feedstock for other chemical reactions, such as the production of a butyl ester from the reaction with carboxylic acid, like acetic acid. The reaction between butadiene and a carboxylic acid to produce butyl esters is described in detail in WO 00/26175.

As described above, t-butyl glycol ether, n-butenyl glycol ether and sec-butenyl glycol ether may have to be recovered from the product stream. This may be carried out using any conventional method, for example, by distillation. Alternatively, this separation step may be carried out by azeotropic distillation. This may require the use of one or more azeotroping agents.

It should be noted that in addition to addition products such as n-butenyl glycol ether, sec-butenyl glycol ether, t-butyl glycol ether, the product stream withdrawn in step b) may also comprise polymerisation by-products such as C₈ olefins (e.g. di-isobutene from isobutene) octatrienes (eg from butadiene+butadiene) and octadienes (e.g. from butadiene and isobutene), and C₁₂ olefins (e.g. from vinyl cyclohexene+butadiene, or C₈ olefin+butadiene). Glyme and diglyme by-products may also be present. For example, where monoethylene glycol ether is employed as the glycol ether feedstock, it may react with butadiene to produce the following by-products: 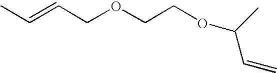 2-butadiene+MEG=crotyl glyme, and 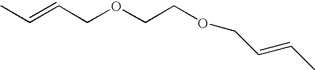 2-butadiene+MEG=crotyl glyme, and 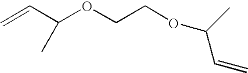 2-butadiene+MEG sec-butenyl glyme.

Similarly, the addition reaction between isobutene, butadiene and monoethylene glycol may produce the following by-products: 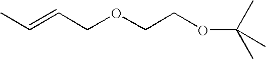 isobutene+butadiene+MEG=crotyl t-butyl glyme, 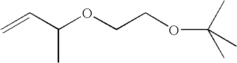 butadiene+isobutene+MEG=t-butyl sec-butenyl glyme, and 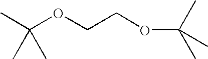 2 isobutene+MEG=t-butyl glyme.

Where diethylene glycol ether is employed as the glycol ether feedstock, on the other hand, the following by-products may be produced: 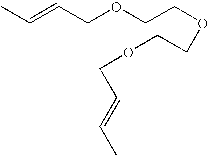 2 butadiene+DEG=crotyl diglyme, 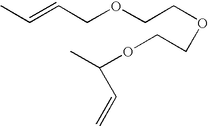 2-butadiene+DEG crotyl sec-butenyl diglyme, 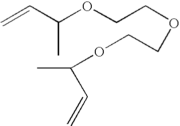 2 butadiene+DEG=sec-butenyl diglyme, 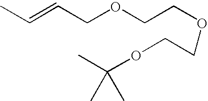 Butadiene+isobutene+DEG=crotyl t-butyl diglyme, 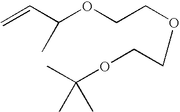 Butadiene+Isobutene+DEG=sec-butenyl t-butyl diglyme, and 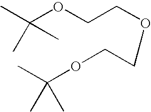 2 isobutene+DEG=t-butyl diglyme.

Such polymerisation and glyme by-products (hereinafter the term is used to include diglyme by-products, unless specifically stated otherwise) may be removed from the product stream, for example, by distillation, or recycled to the addition reactor. Such a recycle can serve to suppress further formation and thereby improve the overall reaction selectivity.

It should be noted that the polymerisation and glyme by-products described above originate either from butadiene alone, or the reaction between butadiene and the glycol reactant employed. In other words, these by-products may be formed when iso-butene is absent from the reactant stream. Accordingly, a second aspect of the invention provides a process for treating a C₄ stream comprising 1,3-butadiene, said process comprising:

a) reacting an aliphatic glycol with said stream in an addition reactor,

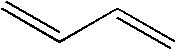

b) withdrawing a product stream comprising sec-butenyl glycol ether, n-butenyl glycol ether and a polymerisation and/or a glyme by-product from the addition reactor, and

c) recovering n-butenyl glycol ether from the product stream, characterised in that

d) polymerisation and/or glyme by-product is recycled to said addition reactor.

In this aspect of the invention, the C₄ stream may consist essentially of 1,3-butadiene, or may be a mixed C₄ stream as described in connection with the first aspect of the present invention.

Recycle of many of the reaction by-products obtained in the process of aspects of the present invention can be advantageous because some of these are under reaction conditions in dynamic equilibrium with the reactants:

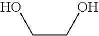

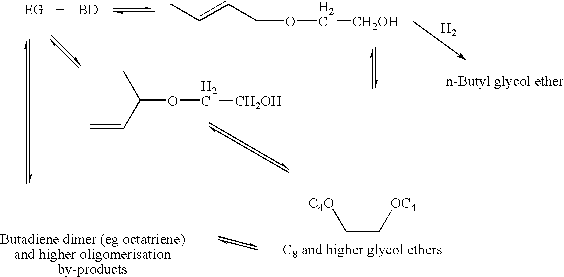

The sulphonic acid catalytic addition of alcohols and glycols aspect of the process of the invention provides advantages including (i) lessening the amount of by-products compared to conventional routes such as e.g. reaction of butanol with an olefin oxide; and (ii) adaptability of the process to produce a variety of n-butyl glycol ethers, including butyl diglycol ether and butyl propylene glycol ether by varying the glycol reactant. Such C₄ butadiene based routes use relatively mild reaction conditions and relatively inexpensive catalysts, and use of soluble di/poly-sulphonic acids avoids the deactivation of heterogeneous catalyst observed due to fouling. Use of di/poly-sulphonic acids also affords higher activity than equivalent acidic hydrogen concentrations for mono-sulphonic acids. This invention also may be used in treatment of C₄ refinery streams for removal of butadiene.

The invention is further illustrated in, but not limited by, the following Examples.

### EXAMPLES

### Example 1

General Procedure A

Reaction of Butadiene with Acetic Acid

Addition reaction of acetic acid to butadiene was conducted in batch mode. A ten-liter stainless steel autoclave equipped with a high efficiency impellor type stirrer and a pressurised butadiene handling facility was used for these experiments. The autoclave had mounted in the form of a stationary annulus around the stirrer a fine mesh stainless steel bag. This was used to contain the catalyst and prevent attrition during stirring and served to facilitate multiple reactions involving the same catalyst charge. The autoclave was also equipped with a sampling valve arrangement which allowed sampling during the course of the reaction.

In a general method used for these reactions, an ion-exchange resin was pre-cleaned of extractible materials by use of a soxhlet extraction apparatus. A range of solvents was used depending on the nature of the resin. For example, with gel type strong acid resins, acetic acid was used and the resin charged to the autoclave in the wet form. For macrorecticular type resins, methanol was used as the solvent and the cleaned resin was then dried in a stream of nitrogen prior to use. This was achieved by stirring in glassware the solution for 16 hours before replacing the resin in the soxhlet extractor and repeating the extraction with methanol or another suitable solvent. The cleaned resin was then dried in a nitrogen stream prior to use. The resin to be tested was then weighed and charged to the stainless steel bag mentioned previously.

The clean autoclave had secured in position the stainless steel bag with the trial catalyst charge. The autoclave was then sealed, pressure tested with nitrogen pressure, and pressure purged of any residual oxygen. The acetic acid feed was subjected to a Karl Fischer water analysis (water level of 0.2% w/w+/−0.05 except where mentioned otherwise). The water level in this feed was modified to the experimental target level by either pre-treatment with acetic anhydride or by adding water. The acetic acid prior to use was also purged with nitrogen to remove dissolved oxygen. The acetic acid charge to the autoclave was used also to help bring into solution and add any inhibitor or other trial additive.

The acetic acid charge was added to the autoclave via a tundish, the autoclave was then pressure purged with nitrogen and heated to the reaction temperature with stirring, at which point the butadiene charge was added to the autoclave as a liquid by forcing the material in from a weighed storage vessel with nitrogen over-pressure. The point of this addition was taken as t=0 minutes and the stirred autoclave contents were sampled at regular intervals and analysed by gas chromatography (equipped with a flame-ionisation detector).

Due to problems associated with analysis due to loss of volatile butadiene from the autoclave samples, it was found to be advantageous to add 0.1-1% w/w on the acetic acid charge of decane as an internal standard for the gas chromatographic (GC) analysis. Control experiments with and without this added decane demonstrated that no significant effect was seen on the reaction itself. The identity of the GC peaks was established by the synthesis of model compounds and GC/MS (MS=mass spectrometry). The GC was calibrated by means of the purchase and synthesis of pure compounds i.e. acetic acid, butenyl acetate, secondary-butenyl acetate, and 4-vinyl cyclohexene. The higher boiling by-products from the reaction were assigned the same response factor determined for butenyl acetate and thereby roughly quantified. All these higher boiling point material peaks were combined together—the higher boiling point materials are collectively referred to as “highers”—and the calculated % w/w used to calculate the reaction selectivity.

General Procedure B

Use of Amberlyst 15H as catalyst without pre-treatment.

The general method described above was used except that Amberlyst 15H resin was used as catalyst in the form as supplied by the manufacturer.

The components charged to autoclave were:

Amberlyst 15H (unwashed) 85 g

Acetic acid 3600 g

1,3-Butadiene 1400 g

Reaction conditions:

60° C. stirring at 1200 rpm

Analysis

The Results are shown in the Table below:

| | Secondary- | | | |
|---|---|---|---|---|
| | butenyl | n-butenyl | 4-vinyl | |
| Runtime | acetate | acetate | cyclohexene | Highers |
| (hours) | (% w/w) | (% w/w) | (% w/w) | (% w/w) |
| 0 | 0 | 0 | 1.3 | 0 |
| 5 | 7.7 | 7.59 | 1.3 | 2.85 |
| 6 | 8.95 | 9.37 | 1.28 | 3.91 |
| 7 | 9.49 | 10.24 | 1.25 | 4.34 |
| 8 | 10.49 | 11.72 | 1.26 | 5.07 |
| 24 | 10.98 | 14.21 | 0.77 | 6.55 |

These results illustrate that the reaction proceeds to give predominately the isomeric C₄ acetates and that some loss of selectivity occurs to higher boiling point materials particularly at longer reaction times. The reaction product was a pale yellow liquid which darkened on standing.

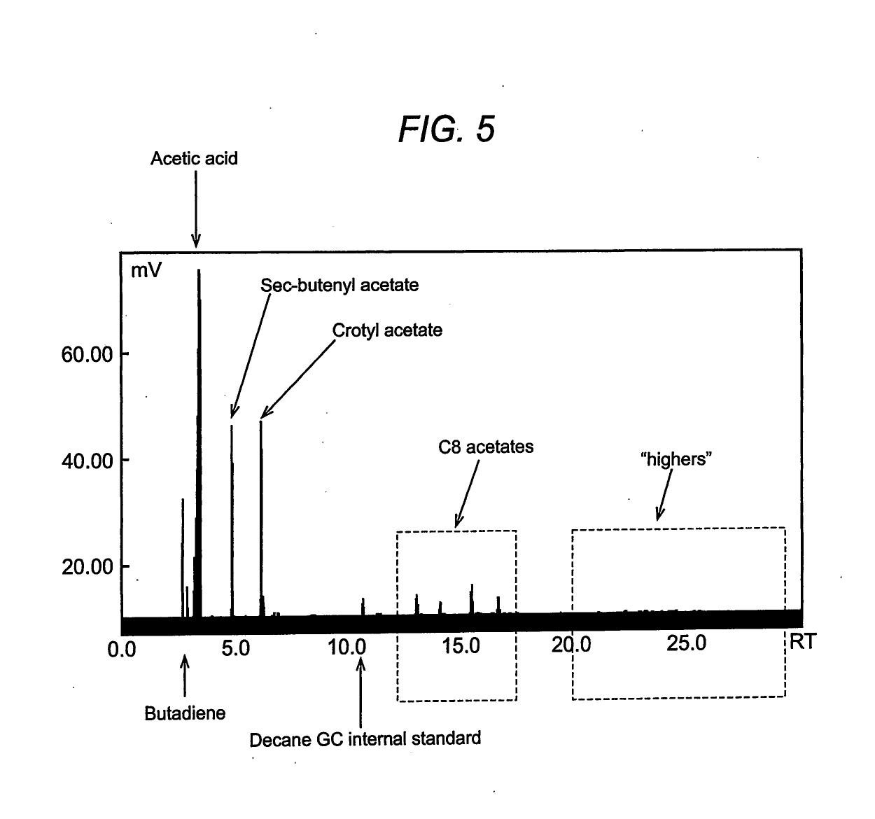

A typical reaction product Gas Chromatogram is shown in FIG. 5.

This illustrates that although the isomeric C₄ acetates are the principal products, a significant loss of selectivity occurs due to the formation of by-product high boiling point materials.

Separation and Identification of Reaction By-products

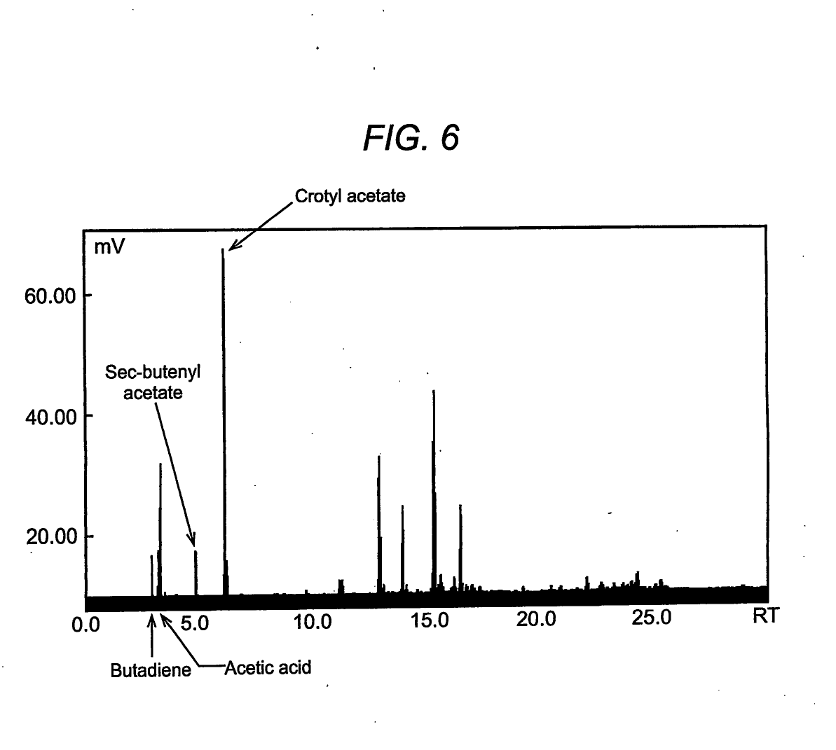

A sample of the reaction product from the addition reaction was concentrated by reduced pressure flash distillation. The resulting residue was analysed by gas chromotography/mass spectroscopy (GC/MS). Analysis of the fragmentation patterns allowed several levels of assignment. These were (i) total carbon number, (ii) presence of acyclic or cyclic/aromatic material, and (iii) presence of an acetate group. The lack of a parent ion did not allow calculation of the molecular formula. FIG. 6 shows the GC of the concentrated by-product mixture. The GC retention time on a CPSIL5 column is strongly related to boiling point. This was confirmed by the mass spectrum results, which indicated that the order of the species on the GC chromatogram are:

Retention Time (minutes)

| 11-16 | 16-20 | 20-25 |
|---|---|---|
| C₈ Acetates | C₁₂ Hydrocarbons | C₁₂ Acetates |

These regions are not absolute—i.e. some C₁₂ hydrocarbons may be retained on the column longer than 20 minutes. These assignments were used for the species in Example 2.

Despite removal of more than 90% of the reaction mixture volume with heating to 90° C. with a 5 fold excess of acetic acid employed in the reaction, some butadiene (a low boiling point compound) remains as an impurity. This demonstrates that butadiene is liberated from the product mixture on heating (with no catalyst being present).

In experiments in which pure samples of crotyl and sec-butenyl acetate are prepared by distillation, butadiene has not been observed. Further, acetic acid has not been found to be formed under these conditions.

Thus it appears that butadiene is being generated from the by-product mixture and not from the C₄ acetates and that the proportion of butadiene is determined by the equilibrium between products and reactants in the reaction medium in which the separated components are maintained.

Recycling of the “higher” by-products in accordance with the process of the present invention, in its preferred aspects, is a particularly convenient manner of controlling the position of the equilibrium, as it allows the proportions of unreacted recycled starting materials and reaction products to be controlled. This provides a means for these “higher” components to drive the reaction dynamic equilibria in Stage (a) of the process of the present invention towards the generation of the desired products as they break down into, for example butadiene, or butadiene and compound Q. Such recycle is accompanied by an overall reduction in the generation of undesirable by-products.

This is in accord with the principle behind the present invention which is that the majority of the reaction by-products have been found to be in equilibrium with the starting materials and the desired reaction products. Thus, the process may be controlled to produce desired products by controlling concentrations of reactants and products. This control may be performed by controlling separated product recycle.

Thus, by separating the reaction product mixture into streams of different composition, including at least one allyl product stream and streams containing other reacted and unreacted products, and subjecting these streams to recycle in selected proportions, selected quantities of the various components of the product mixture may be obtained. For example, components of at least a portion of at least one of said separated streams from step (a) is subjected to reaction conditions under which (i) C₄-C₁₀ conjugated diene, (ii) compound Q and (iii) allyl addition product participate in an equilibrium reaction:
(i)+(ii) [symbol] (iii)

The amounts of components (i), (ii), and (iii) in this reaction mixture may be controlled by adjusting the size of the portion of at least one of the separated streams that is subjected to these reaction conditions.

### Example 2

Example 2 was carried out as described in Example 1, except washed resin was used. The reaction was continued until the major components had to come to equilibrium (i.e., little or no change with time in amount was observed by GC). At this point the mixture was analysed, the stirrer switched off, and a portion of the butadiene removed from the equilibrated mixture by controlled venting of the gas phase. The vent line was closed, stirring resumed, and the reaction time taken as t=0. The stirring of the reaction mixture was by a high efficiency gas turbine type impellor which gives fast physical equilibration of gas and vapour space species. Liquid samples were then taken to follow the relaxation of the system towards a new chemical equilibrium. The gathered GC data is tabulated below. The values are expressed as moles/L. For sec-butenyl/crotyl acetate, acetic acid and butadiene, samples of pure compounds were used for GC calibration. The response factor for a model compound crotyl acetate was used for the C₈ dimers, C₈ acetates, C12 trimers and oligomers and this allowed an approximate concentration in moles/litre to be estimated for these mixtures of compounds.

| | Sec- | | | | | | | |
|---|---|---|---|---|---|---|---|---|
| Time | Butenyl | n-Butenyl | C₈ BD | C₈ | C₁₂ BD | | Acetic | |
| (mins) | Acetate | Acetate | dimers | acetates | trimers | oligomers | acid | Butadiene |
| 4 | 0.524 | 0.6843 | 0.0219 | 0.119 | 0.0642 | 0.0285 | 10.933 | 0.733 |
| 36 | 0.522 | 0.6604 | 0.0221 | 0.113 | 0.0596 | 0.0284 | 10.965 | 0.783 |
| 66 | 0.522 | 0.6622 | 0.0221 | 0.115 | 0.0603 | 0.0293 | 10.961 | 0.771 |
| 104 | 0.522 | 0.6620 | 0.0223 | 0.115 | 0.0646 | 0.0287 | 10.961 | 0.760 |
| 153 | 0.518 | 0.6601 | 0.0224 | 0.117 | 0.0618 | 0.0296 | 10.966 | 0.767 |
| 1229 | 0.509 | 0.6451 | 0.0216 | 0.113 | 0.0612 | 0.0285 | 10.994 | 0.808 |
| 1319 | 0.501 | 0.6406 | 0.0219 | 0.113 | 0.0616 | 0.0275 | 11.006 | 0.823 |
| 1437 | 0.501 | 0.6393 | 0.0217 | 0.111 | 0.0619 | 0.0264 | 11.009 | 0.832 |
| 1557 | 0.499 | 0.6388 | 0.0216 | 0.112 | 0.0617 | 0.0274 | 11.011 | 0.829 |
| 1678 | 0.499 | 0.6414 | 0.0206 | 0.114 | 0.0617 | 0.0287 | 11.006 | 0.819 |
| 2683 | 0.490 | 0.6280 | 0.0217 | 0.111 | 0.0630 | 0.0281 | 11.031 | 0.844 |
| 2783 | 0.483 | 0.6222 | 0.0215 | 0.111 | 0.0612 | 0.0290 | 11.044 | 0.858 |
| 2914 | 0.485 | 0.6253 | 0.0215 | 0.109 | 0.0601 | 0.0269 | 11.042 | 0.872 |
| 3021 | 0.482 | 0.6212 | 0.0218 | 0.110 | 0.0594 | 0.0268 | 11.047 | 0.878 |
| 3122 | 0.482 | 0.6189 | 0.0200 | 0.108 | 0.0587 | 0.0265 | 11.052 | 0.891 |
| 4108 | 0.477 | 0.6158 | 0.0218 | 0.108 | 0.0605 | 0.0264 | 11.060 | 0.892 |
| 4219 | 0.470 | 0.6062 | 0.0217 | 0.107 | 0.0602 | 0.0273 | 11.077 | 0.906 |
| 4329 | 0.469 | 0.6083 | 0.0219 | 0.108 | 0.0604 | 0.0276 | 11.075 | 0.901 |

These results show that the addition reaction products and reactants are in chemical equilibria. Thus, removal of butadiene from the liquid phase by venting off butadiene in the vapour phase results in the reverse of the addition reaction. For example, as the level of crotyl acetate and C₈ acetate decreases, an increase in the amount of acetic acid and butadiene is observed.

### Example 3

Following the procedure of Example 2, a portion of the reaction mixture is withdrawn and is subjected to fractional distillation to separate a butadiene-rich fraction, a crotyl acetate-rich fraction and a sec-butyl acetate-rich fraction. The butadiene-rich fraction is recycled to the reaction mixture, and depending upon which product is to be recovered, the crotyl acetate rich fraction or the sec-butyl acetate-rich fraction (i.e. the product which is not required to be recovered) is recycled to the reaction mixture. The overall process thus provides an integrated process to produce either or both of crotyl acetate and sec-butyl acetate. The recovered products are then converted to desired product (e.g. butyraldehyde or methylethylketone) by subjecting the product to one more finishing steps selected from hydrolysis, hydrogenation, isomerization, and cracking. Butyraldehyde is produced by hydrolysis of crotyl ester followed by catalytic isomerization. MEK is produced by hydrolysis of sec-butenyl ester followed by catalytic isomerization.

Alternatively, both butyraldehyde and MEK may be co-produced in predetermined proportions by withdrawing both crotyl and sec-butenyl esters in a predetermined proportion, converting the separated streams to desired products, and controlling crotyl and/or sec-butenyl ester recycle streams to the reactor.

1: An integrated chemical process comprising
(a) combining a hydrocarbon stream comprising butadiene with a compound Q selected from compounds defined as:
R¹(CO)ₙ—OH
wherein n is 1 or 0, and
R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups,
under addition reaction conditions to form a reaction mixture containing at least a crotyl addition product and a sec-butenyl addition product;
(b) separating the reaction product mixture into streams comprising a crotyl product containing stream, a sec-butenyl containing product stream, and at least one stream containing other reacted and unreacted products;
(c) controlling the proportion of the product streams by recycling a portion or all of a separated crotyl product stream and/or a sec-butenyl product stream and other product streams to the addition reactor;
(d) subjecting one or more separated product streams to one or more process selected from hydrolysis, hydrogenation, isomerization, and cracking to form product derivatives in preselected proportions; and
(e) recovering one or more resulting product derivatives.

2: The process of claim 1 wherein Q is selected from carboxylic acids containing 1 to 6 carbon atoms, monohydric alcohols containing 1 to 10 carbon atoms, and dihydric alcohols containing 2 to 10 carbon atoms.

3: The process of claim 1 wherein Q is selected from acetic acid, methanol, and ethanol.

4: The process of claim 1 wherein the recycle stream comprises at least one or more by-product derived from butadiene dimerisation or oligomerisation or reaction of such dimerisation or oligomerisation with compound Q.

5: The process of claim 1 in which butyraldehyde and methyl ethyl ketone are co-produced in controlled proportions by formation of a crotyl ester or ether and a sec-butenyl ester or ether by catalytic addition of a carboxylic acid or alcohol to butadiene in an addition reactor, separation of at least a portion of crotyl ester or ether and a portion of sec-butenyl ester or ether, recycling the remaining products to the addition reactor to control the desired amount of crotyl and sec-butenyl ester or ether, converting the separated crotyl ester or ether to butyraldehyde by isomerization and hydrolysis, and converting the separated sec-butenyl ester or ether to methyl ethyl ketone by isomerization and hydrolysis.

6: The process of claim 5 in which Q is acetic acid and sec-butenyl acetate is selectively and controllably recycled to the addition reactor to form a greater proportion of crotyl acetate, which is converted to butyraldehyde.

7: The process of claim 5 in which allyl ether derivatives are formed which are isomerised to an enol ether using a strong base.

8: The process of claim 5 in which at least one of butyraldehyde or methyl ethyl ketone is hydrogenated to a corresponding alcohol.

9: The process of claim 1 in which butanol and butyl carboxylate are co-produced in controlled proportions by formation of a crotyl ester and a sec-butenyl ester by catalytic addition of a carboxylic acid to butadiene in an addition reactor, separation of at least a portion of the crotyl ester and recycling the remaining products to the addition reactor to control the desired amount of crotyl ester, converting the separated crotyl ester to butyl carboxylate by hydrogenation and conversion of at least a portion of such butyl carboxylate to butanol and carboxylic acid by hydrolysis and recycling of the carboxylic acid to the addition reactor.

10: The process of claim 1 in which butyraldehyde and butyl carboxylate are co-produced in controlled proportions by formation of a crotyl ester and a sec-butenyl ester by catalytic addition of a carboxylic acid to butadiene in an addition reactor, separation of at least a portion of the crotyl ester and recycling the remaining products to the addition reactor to control the desired amount of crotyl ester, converting the separated crotyl ester to butyraldehyde and carboxylic acid by isomerisation and hydrolysis and recycling of the carboxylic acid to the addition reactor.

11: The process of claim 9 in which the carboxylic acid is acetic acid.

12: The process of claim 1 in which the butadiene-containing hydrocarbon stream comprises a C₄ refinery stream.

13: The process of claim 1 in which a portion of the crotyl product and/or a sec-butenyl product is cracked to butadiene and compound Q.

14: The process of claim 1 in which Q is a glycol of the formula:
HO(CHR′CHR″O)ₙH
wherein R′ and R″ are each independently hydrogen or a hydrocarbyl group having up to 10 carbons atoms, and n is at least 1.

15: The process of claim 14 in which Q is monoethylene glycol or diethylene glycol.

16: An integrated chemical process comprising
(a) combining a hydrocarbon stream comprising a C₄-C₁₀ conjugated diene with a compound Q selected from compounds defined as:
R¹(CO)ₙ—OH
wherein n is 1 or 0, and
R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups,
under addition reaction conditions to form a reaction mixture containing at least one allyl addition product;
(b) separating the reaction product mixture into streams comprising at least one allyl product stream, and at least one stream containing other reacted and unreacted products;
(c) controlling the proportion of the product streams by recycling a portion or all of a separated allyl product stream and other product streams to the addition reactor;
(d) subjecting one or more separated product streams to one or more process selected from hydrolysis, hydrogenation, isomerization, and cracking to form product derivatives in preselected proportions; and
(e) recovering one or more resulting product derivatives.

17: The process of claim 16 in which the conjugated diene is 1,3-butadiene, 1,3-pentadiene, or 2-methyl-1,3-butadiene.

18: The process of claim 16 in which the conjugated diene is 1,3-butadiene.

19: The process of claim 16 in which the addition reaction is catalysed by a homogeneous sulphonic acid catalyst containing at least two sulphonic acid groups.

20: The process of claim 1 in which one or more streams containing isobutene, raffinate 1 and raffinate 2 are isolated.

21: A process for producing at least one product stream of selected composition, comprising:
(a) combining a hydrocarbon stream comprising a C₄-C₁₀ conjugated diene with a compound Q selected from compounds defined as:
R¹(CO)ₙ—OH
wherein n is 1 or 0, and
R¹ is a C₁-C₂₀ alkyl or a C₂-C₂₀ alkenyl group or R¹ is a C₆-C₁₀ aryl group or a C₇-C₁₁ aralkyl group, which may be unsubstituted or independently substituted by hydroxy and C₁-C₂₀ alkoxy and alkyl hydroxy ether groups,
under addition reaction conditions to form a reaction mixture containing at least one allyl addition product;
(b) separating the reaction product mixture into streams comprising at least one allyl product stream, and at least one stream containing other reacted and unreacted products;
(c) maintaining a reaction mixture, wherein components of at least at least a portion of at least one of said separated streams from step (a) is subjected to reaction conditions under which (i) C₄-C₁₀ conjugated diene, (ii) compound Q and (iii) allyl addition product participate in an equilibrium reaction
(i)+(ii) [symbol] (iii);
(d) controlling the amount of components (i), (ii) and (iii) in said reaction mixture by adjusting the size of said portion of at least one of said separated streams that is subjected to the reaction conditions of step (c);
(e) recovering at least one component of the separated streams from step (a) and of the reaction mixture of step (c); and
(f) optionally, subjecting one or more recovered components to one or more process selected from hydrolysis, hydrogenation, isomerization, and cracking to form product derivatives in preselected proportions.

22: The process of claim 21 in which the conjugated diene is 1,3-butadiene, 1,3-pentadiene, or 2-methyl-1,3-butadiene.

23: The process of claim 21 in which the conjugated diene is 1,3-butadiene.

24: The process of claim 21 in which the conjugated diene is 1,3-butadiene, n=1, and R¹ is methyl.

25: The process of claim 24 in which the product streams comprise crotyl ester and sec-butenyl ester and at least one of such streams is converted by hydrolysis and isomerization.

26: The process of claim 16 in which one or more streams containing isobutene, raffinate 1 and raffinate 2 are isolated.

27: The process of claim 10 in which the carboxylic acid is acetic acid.
